(19) United States
(12) Patent Application Publication
(10) Pub. No.: US 2024/0269145 A1
(43) Pub. Date: Aug. 15, 2024

(54) USE OF INDOLE COMPOUND AS INHIBITORS OF FERROPTOSIS

(71) Applicant: MITOIMMUNE THERAPEUTICS INC., Seoul (KR)

(72) Inventors: Soon Ha Kim, Seoul (KR); Hye Kyung Chang, Seoul (KR); Mooyoung Seo, Seoul (KR); Jeong-Hee Yang, Seoul (KR); Hyunjun Park, Seoul (KR); Jeong Hyang Park, Seoul (KR); Yun Min Park, Seoul (KR); Jin Sung Park, Seoul (KR); Jae Young Kim, Seoul (KR); Yewon Yun, Seoul (KR)

(21) Appl. No.: 18/422,484

(22) Filed: Jan. 25, 2024

(30) Foreign Application Priority Data
Jan. 27, 2023 (KR) 10-2023-0011082

(51) Int. Cl. A61K 31/541; A61K 31/404; A61K 31/454; A61K 31/496; A61K 31/5377; A61P 43/00

(52) U.S. Cl. CPC A61K 31/541; A61K 31/404; A61K 31/454; A61K 31/496; A61K 31/5377; A61P 43/00

(57) ABSTRACT

The present invention relates to a use of a compound of Chemical Formula 1, an isomer thereof, a solvate thereof, a hydrate thereof, or a pharmaceutically acceptable salt thereof as a ferroptosis inhibitor, and a method of inhibiting ferroptosis using the same. The compound of Chemical Formula 1 according to the present invention may exhibit an effect of inhibiting ferroptosis in various cells such as cardiac, renal, nerve, retinal, hepatic or pulmonary cells, and thus it may be effectively used in preventing or treating a ferroptosis-related disease.

### CROSS-REFERENCE TO RELATED APPLICATION

This application claims priority to and the benefit of Korean Patent Application No. 2023-0011082, filed on Jan. 27, 2023, the disclosure of which is incorporated herein by reference in its entirety.

### BACKGROUND

### 1. Field of the Invention

The present invention relates to a use of a compound of Chemical Formula 1, an isomer thereof, a solvate thereof, a hydrate thereof, or a pharmaceutically acceptable salt thereof as a ferroptosis inhibitor, and a method of inhibiting ferroptosis using the same.

### 2. Discussion of Related Art

Ferroptosis is cell death caused by iron-dependent lipid peroxidation, recently classified and named as regulated necrotic cell death (RNCD).

Ferroptosis is a type of cell death different from apoptosis, necroptosis, and pyroptosis, and it exhibits morphological, biochemical, and genetic differences. A typical characteristic observed in cells undergoing ferroptosis is peroxidation of lipids present in the cell membrane, and lipid peroxidation causes the cell membrane to collapse and induces cell death. Cells generally operate an antioxidant system in which cystine is supplied through system Xc to maintain the concentration of glutathione and glutathione peroxidase 4 (GPX4) is activated to remove lipid peroxides in cells. However, when the glutathione concentration is low or GPX4 does not function, damage to lipids, proteins, nucleic acids, or the like and cell membrane collapse are induced due to the accumulation of lipid peroxides, resulting in ferroptosis. It is known that another factor involved in the accumulation of lipid peroxides is lipid reactive oxygen species (ROS), which are generated when iron ions are coupled with cytosolic ROS through the Fenton reaction.

It is known that reactive oxygen species are produced in mitochondria, which is an intracellular organelle, and many studies on the relationship between mitochondria and ferroptosis have recently been published. Accordingly, many researchers regard the occurrence of lipid peroxidation reactive oxygen species and iron accumulation as factors that differentiate ferroptosis from other types of cell death, and use them as representative biomarkers along with reactive oxygen species.

The phenomenon of ferroptosis has been reported in various pathological situations, and inhibition of ferroptosis can be considered very important in disease prevention and treatment. Representative diseases in which ferroptosis occurs include ischemic diseases (e.g. myocardial infarction, stroke, renal infarction), neurodegenerative diseases, inflammatory diseases, infectious diseases, ocular diseases (e.g. retinal degeneration, macular degeneration), autoimmune diseases, and lung diseases, and its association has been reported in various diseases. Accordingly, mechanistic research to understand ferroptosis, which is a phenomenon commonly found in various diseases, and the discovery and development of ferroptosis inhibitors for disease prevention and treatment are actively underway.

Since ferroptosis was discovered and named, ferroptosis inhibitors have been developed by many researchers, and representative examples are ferrostatin-1 (fer-1), UAMC-3203, and liproxtatin-1 (liprox-1), which are known to inhibit lipid peroxidation as an antioxidant (radical trapping agent; RTA). In addition, many studies are in progress on the role of known antioxidants as ferroptosis inhibitors. However, the above inhibitors are still in development, and the development of ferroptosis inhibitors exhibiting excellent efficacy is still an urgent task.

The present inventors conducted a study to determine whether the composition of the present invention exhibits pharmaceutically significant efficacy against ferroptosis, which is regulated cell death, and confirmed its effectiveness.

### RELATED ART DOCUMENT

### Patent Document

International Patent Publication WO 2009/025478

### SUMMARY OF THE INVENTION

The present inventors completed the present invention by discovering that a compound of Chemical Formula 1, an isomer thereof, a solvate thereof, a hydrate thereof, or a pharmaceutically acceptable salt thereof effectively inhibits ferroptosis.

Accordingly, the present invention is directed to providing a pharmaceutical composition for inhibiting ferroptosis, comprising a compound of Chemical Formula 1, an isomer thereof, a solvate thereof, a hydrate thereof, or a pharmaceutically acceptable salt thereof as an active ingredient.

In addition, the present invention is directed to providing a method of inhibiting ferroptosis and a method of inhibiting cytosolic accumulation of iron ions, comprising administering a compound of Chemical Formula 1, an isomer thereof, a solvate thereof, a hydrate thereof, or a pharmaceutically acceptable salt thereof to a subject in need thereof in a pharmaceutically effective amount.

In addition, the present invention is directed to providing a method of reducing cytosolic or mitochondrial reactive oxygen species (ROS), particularly lipid ROS, comprising administering a compound of Chemical Formula 1, an isomer thereof, a solvate thereof, a hydrate thereof, or a pharmaceutically acceptable salt thereof to a subject in need thereof in a pharmaceutically effective amount.

### BRIEF DESCRIPTION OF THE DRAWINGS

FIG. 1 shows a graph of an effect of Example Compound 1 on inhibiting cytosolic lipid reactive oxygen species after RSL3 treatment in MLE-12 cells according to Experimental Example 3-1.

FIG. 2 shows a graph of an effect of Example Compound 1 on inhibiting cytosolic lipid reactive oxygen species after erastin treatment in HT-22 cells according to Experimental Example 3-2.

FIG. 3 shows a graph of an effect of Example Compound 1 on inhibiting cytosolic lipid reactive oxygen species after erastin treatment in NIH/3T3 cells according to Experimental Example 3-3.

FIG. 4 shows a graph of an effect of Example Compound 1 on inhibiting cytosolic lipid reactive oxygen species after RSL3 treatment in PC-12 cells according to Experimental Example 3-4.

FIG. 5 shows a graph of an effect of Example Compound 1 on inhibiting cytosolic lipid reactive oxygen species after RSL3 treatment in ARPE-19 cells according to Experimental Example 3-5.

FIG. 6 shows a graph of an effect of Example Compound 1 on inhibiting cytosolic reactive oxygen species after RSL3 treatment in MLE-12 cells according to Experimental Example 4.

FIG. 7 shows a graph of an effect of Example Compound 1 on inhibiting mitochondrial reactive oxygen species after RSL3 treatment in MLE-12 cells according to Experimental Example 5.

FIG. 8 shows a graph of an effect of Example Compound 1 on inhibiting cytosolic iron ion accumulation after RSL3 treatment in MLE-12 cells according to Experimental Example 6.

FIG. 9 shows a set of images of an effect of Example Compound 1 on inhibiting cytosolic iron ion accumulation after RSL3 treatment in PC-12 cells and erastin treatment in H9C2 cells according to Experimental Example 7.

FIG. 10 shows a graph of an effect of Example Compound 1 on inhibiting mitochondrial iron ion accumulation after RSL3 treatment in MLE-12 cells according to Experimental Example 8.

FIG. 11 shows a set of images of an effect of Example Compound 1 on inhibiting mitochondrial iron ion accumulation after RSL3 treatment in MLE-12 cells and PC-12 cells according to Experimental Example 9-1.

FIG. 12 shows a set of images of an effect of Example Compound 1 on inhibiting mitochondrial iron ion accumulation after erastin treatment in H9C2 cells according to Experimental Example 9-2.

FIG. 13 shows a set of images of an effect of Example Compound 1 on protecting against cell death after erastin treatment in H9C2 cells according to Experimental Example 10.

FIG. 14 shows a set of images of an effect of Example Compound 1 on inhibiting morphological changes in mitochondria after erastin treatment in H9C2 cells according to Experimental Example 11.

### DETAILED DESCRIPTION OF EXEMPLARY EMBODIMENTS

Hereinafter, the present invention will be described in detail.

Meanwhile, each description and embodiment disclosed in the present application may also be applied to other descriptions and embodiments. That is, all combinations of the various elements disclosed in the present application fall within the scope of the present invention. In addition, the scope of the present invention is not limited by the specific description described below.

When a part is said to “include”, “comprise” a certain component, unless specified otherwise, this means that it may further include other components, rather than exclude other components.

The present invention provides a composition for inhibiting ferroptosis, comprising, as an active ingredient, a compound of Chemical Formula 1 or a pharmaceutically acceptable salt thereof.

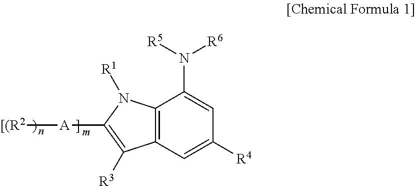

wherein,

n is an integer from 1 to 3,

m is 0 or 1,

A represents phenyl,

R¹ is hydrogen, or a C₁-C₆ alkyl,

R² represents hydrogen, a halogen or a C₁-C₆ alkoxy, or represents hydroxy-C₁-C₆ alkyl, —(CH₂)ₚCO₂R⁷, —NHR⁸, —N(H)S(O)₂R⁷ or —NHC(O)R⁷, wherein p is an integer from 0 to 3, R⁷ represents hydrogen or a C₁-C₃ alkyl, and R⁸ represents (C₁-C₃ alkyl)piperidinyl, or (C₁-C₃ alkyl)sulfonyl,

R³ represents hydrogen, a halogen, a C₁-C₆ alkyl or phenyl, or represents —(CH₂)p-heterocycle in which the heterocycle is a 5- to 6-membered ring containing one or two heteroatom(s) selected among S, N and O atoms, wherein p is an integer from 0 to 3, provided that R³ is phenyl when m is 0,

R⁴ represents a halogen, a C₁-C₆ alkyl, hydroxy-C₁-C₆ alkyl, —O-phenyl, —(CH₂)ₚCO₂R⁷, —(CH₂)p-heterocycle in which the heterocycle is a 5- to 6-membered ring containing one or two heteroatom(s) selected among S, N and O atoms, or proline-N-carbonyl, wherein p is an integer from 0 to 3, and R⁷ is as described above,

R⁵ is hydrogen, or a C₁-C₆ alkyl, and

R⁶ represents a C₁-C₆ alkyl, a C₃-C₆ cycloalkyl, a heterocycle or heterocyclyl-C₁-C₆ alkyl, wherein the heterocycle is a 3- to 8-membered ring including 1 to 3 heteroatom(s) selected among S, N and O atoms, and R⁶ may be substituted with (C₁-C₆ alkyl)amine, hydroxy-C₁-C₆ alkyl, or (C₁-C₆ alkyl)sulfonyl.

The compound of Chemical Formula 1 of the present invention may be used in the form of a pharmaceutically acceptable salt thereof. In particular, the pharmaceutically acceptable salt may be an acid addition salt formed by a free acid. Here, the acid addition salt may be obtained from an inorganic acid such as hydrochloric acid, nitric acid, phosphoric acid, sulfuric acid, hydrobromic acid, hydroiodic acid, nitrous acid, and phosphorous acid, a non-toxic organic acid such as aliphatic mono and dicarboxylates, phenyl-substituted alkanoates, hydroxy alkanoates and alkanedionates, aromatic acids, and aliphatic and aromatic sulfonic acid, and an organic acid such as trifluoroacetic acid, acetate, benzoic acid, citric acid, lactic acid, maleic acid, gluconic acid, methanesulfonic acid, 4-toluenesulfonic acid, tartaric acid, and fumaric acid. Types of such pharmaceutically acceptable salts may include sulfates, pyrosulfates, bisulfates, sulfites, bisulfites, nitrites, phosphates, monohydrogen phosphates, dihydrogen phosphates, metaphosphates, pyrophosphate chlorides, bromides, iodides, fluorides, acetates, propionates, and the like

The composition of the present invention may comprise not only the compound of Chemical Formula 1 or a pharmaceutically acceptable salt thereof, but also all salts, isomers, hydrates and/or solvates thereof that may be prepared by typical methods.

As used herein, the “isomer” may refer to a compound of the invention, which has the same chemical or molecular formula but is structurally or sterically different, or a salt thereof. Such isomers include all of a structural isomer such as a tautomer, an R or S isomer having an asymmetric carbon center, an isomer such as a geometric isomer (trans, cis), and an optical isomer (enantiomer). All of these isomers and mixtures thereof are also included within the scope of the present invention.

As used herein, the “hydrate” may refer to a compound of the present invention including a stoichiometric or non-stoichiometric amount of water bonded by a non-covalent intermolecular force, or a salt thereof. The hydrate of the compound represented by Chemical Formula 1 of the present invention may include a stoichiometric or non-stoichiometric amount of water bonded by a non-covalent intermolecular force. The hydrate may contain at least 1 equivalent, preferably 1 to 5 equivalents of water. Such a hydrate may be prepared by crystallizing the compound represented by Chemical Formula 1 of the present invention, an isomer thereof, or pharmaceutically acceptable salt thereof from water or a solvent containing water.

As used herein, the “solvate” may refer to a compound of the present invention including a stoichiometric or non-stoichiometric amount of solvent bonded by a non-covalent intermolecular force, or a salt thereof. Preferred solvents in this regard include volatile, non-toxic, and/or solvents suitable for administration to humans.

As used herein, the term ‘alkyl’ refers to an aliphatic hydrocarbon radical. The alkyl may be either a “saturated alkyl” containing no alkenyl or alkynyl moiety or an “unsaturated alkyl” containing at least one alkenyl or alkynyl moiety, and may have 1 to 20 carbon atoms, unless otherwise defined.

The term ‘alkylene’ refers to a divalent hydrocarbon group in which a radical is additionally formed from the alkyl, and examples include methylene, ethylene, propylene, butylene, and isobutylene and the like, but are not limited thereto.

The term ‘alkoxy’ refers to an alkyl-oxy having 1 to 10 carbon atoms, unless otherwise defined.

The term ‘cycloalkyl’ refers to a saturated aliphatic 3- to 10-membered ring, unless otherwise defined. Typical cycloalkyl groups include cyclopropyl, cyclobutyl, cyclopentyl, cyclohexyl, and the like, but are not limited thereto.

Unless otherwise defined, the term ‘heterocycle’ refers to a 3- to 10-membered ring, which includes 1 to 3 heteroatoms selected from the group consisting of N, O and S, may be fused with benzo or a C₃-C₈ cycloalkyl, and is saturated or includes one or two double bond(s), preferably a 4- to 8-membered ring, and more preferably a 5- to 6-membered ring. Further, the term may be used interchangeably with the term ‘heterocyclyl.’ Examples of the heterocycle include pyrroline, pyrrolidine, imidazoline, imidazolidine, pyrazolidine, pyran, piperidine, morpholine, thiomorpholine, piperazine, hydrofuran, and the like, but are not limited thereto.

All other terms and abbreviations used herein, unless otherwise defined, are to be interpreted as commonly understood by a person with ordinary skill in the art to which this invention pertains.

In an exemplary embodiment of the present invention, in the compound of Chemical Formula 1,

R³ represents hydrogen, a halogen, or phenyl, or represents —(CH₂)p-heterocycle in which the heterocycle is morpholino or piperazinonyl, wherein p is an integer from 0 to 1, provided that R³ may be phenyl when m is 0.

In an exemplary embodiment of the present invention, in the compound of Chemical Formula 1,

R⁴ is a halogen, a C₁-C₃ alkyl, hydroxy-C₁-C₃ alkyl, —O-phenyl, —(CH₂)ₚCO₂-ethyl, —(CH₂)p-heterocycle in which the heterocycle is thiomorpholino, morpholino, piperazinonyl, or pyrrolidinyl, or proline-N-carbonyl, wherein p may be an integer from 0 to 1.

In an exemplary embodiment of the present invention, in the compound of Chemical Formula 1,

R⁵ is hydrogen, or a C₁-C₃ alkyl, and

R⁶ represents a C₁-C₃ alkyl, a C₃-C₆ cycloalkyl, a heterocycle or heterocyclyl-C₁-C₃ alkyl, wherein the heterocycle may be tetrahydro-2H-pyran, or piperidinyl, and when R⁶ is a heterocycle or heterocyclyl-C₁-C₃ alkyl, R⁶ may be substituted with (C₁-C₆ alkyl)amine, hydroxy-C₁-C₆ alkyl, or (C₁-C₆ alkyl)sulfonyl.

In the present invention, examples of the compound of Chemical Formula 1 comprise Compounds 1 to 32 listed in the following Table 1, or pharmaceutically acceptable salts thereof.

TABLE 1

| Cmpd | | |
|---|---|---|
| # | Structure | Chemical name |
| 1 | 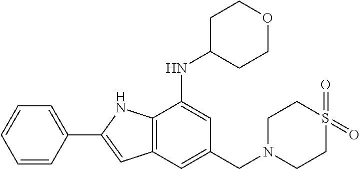 [SMILES: O=S1(=O)CCN(Cc2cc(NC3CCOCC3)c3[nH]c(-c4ccccc4)cc3c2)CC1] | 5-[(1,1-dioxido-4-thiomorpholinyl)methyl]-2- phenyl-N-(tetrahydro-2H-pyran-4-yl)-1H-indole-7- amine |
| 2 | 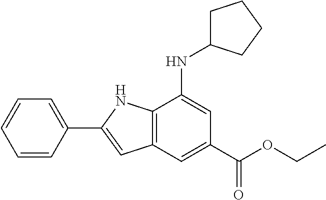 [SMILES: CCOC(=O)c1cc(NC2CCCC2)c2[nH]c(-c3ccccc3)cc2c1] | ethyl 7-(cyclopentylamino)-2-phenyl-1H-indole-5- carboxylate |
| 3 | 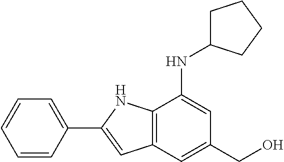 [SMILES: OCc1cc(NC2CCCC2)c2[nH]c(-c3ccccc3)cc2c1] | (7-(cyclopentylamino)-2-phenyl-1H-indole-5- yl)methanol |
| 4 | 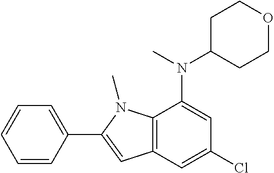 [SMILES: CN(c1cc(Cl)cc2cc(-c3ccccc3)n(C)c12)C1CCOCC1] | 5-chloro-N,1-dimethyl-2-phenyl-N-(tetrahydro- 2H-pyran-4-yl)-1H-indole-7-amine |
| 5 | 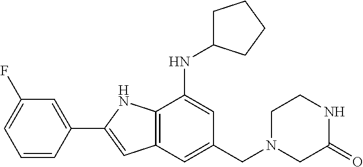 [SMILES: O=C1CN(Cc2cc(NC3CCCC3)c3[nH]c(-c4cccc(F)c4)cc3c2)CCN1] | 4-((7-(cyclopentylamino)-2-(3-fluorophenyl)-1H- indol-5-yl)methyl)piperazine-2-one |
| 6 | 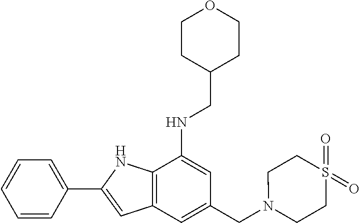 [SMILES: O=S1(=O)CCN(Cc2cc(NCC3CCOCC3)c3[nH]c(-c4ccccc4)cc3c2)CC1] | 4-((2-phenyl-7-(((tetrahydro-2H-pyran-4- yl)methyl)amino)-1H-indol-5- yl)methyl)thiomorpholine 1,1-dioxide |
| 7 | 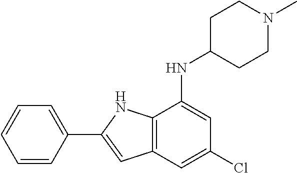 [SMILES: CN1CCC(Nc2cc(Cl)cc3cc(-c4ccccc4)[nH]c23)CC1] | 5-chloro-N-(1-methylpiperidin-4-yl)-2-phenyl-1H- indole-7-amine |
| 8 | 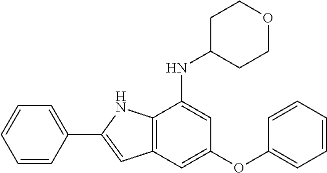 [SMILES: c1ccc(Oc2cc(NC3CCOCC3)c3[nH]c(-c4ccccc4)cc3c2)cc1] | 5-phenoxy-2-phenyl-N-(tetrahydro-2H-pyran-4- yl)-1H-indole-7-amine |
| 9 | 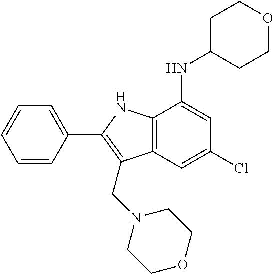 [SMILES: Clc1cc(NC2CCOCC2)c2[nH]c(-c3ccccc3)c(CN3CCOCC3)c2c1] | 5-chloro-3-(morpholinomethyl)-2-phenyl-N- (tetrahydro-2H-pyran-4-yl)-1H-indole-7-amine |
| 10 | 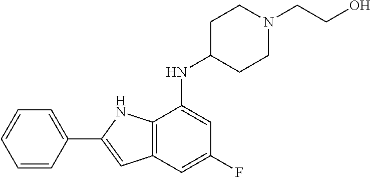 [SMILES: OCCN1CCC(Nc2cc(F)cc3cc(-c4ccccc4)[nH]c23)CC1] | 2-(4-((5-fluoro-2-phenyl-1H-indol-7- yl)amino)piperidin-1-yl)ethan-1-ol |
| 11 | 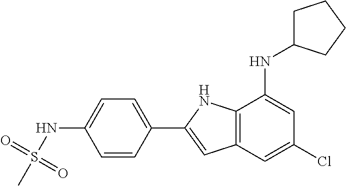 [SMILES: CS(=O)(=O)Nc1ccc(-c2cc3cc(Cl)cc(NC4CCCC4)c3[nH]2)cc1] | N-(4-(5-chloro-7-(cyclopentylamino)-1H-indole-2- yl)phenyl)methanesulfonamide |
| 12 | 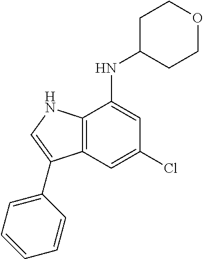 [SMILES: Clc1cc(NC2CCOCC2)c2[nH]cc(-c3ccccc3)c2c1] | 5-chloro-3-phenyl-N-(tetrahydro-2H-pyran-4-yl)- 1H-indole-7-amine |
| 13 | 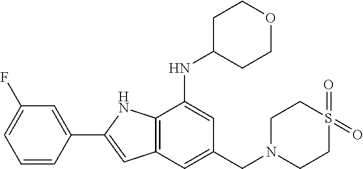 [SMILES: O=S1(=O)CCN(Cc2cc(NC3CCOCC3)c3[nH]c(-c4cccc(F)c4)cc3c2)CC1] | 4-((2-(3-fluorophenyl)-7-((tetrahydro-2H-pyran-4- yl)amino)-1H-indol-5-yl)methyl)thiomorphline 1,1-dioxide |
| 14 | 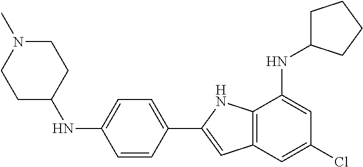 [SMILES: CN1CCC(Nc2ccc(-c3cc4cc(Cl)cc(NC5CCCC5)c4[nH]3)cc2)CC1] | 5-chloro-N-cyclopentyl-2-(4-((1-methylpiperidin- 4-yl)amino)phenyl)-1H-indole-7-amine |
| 15 | 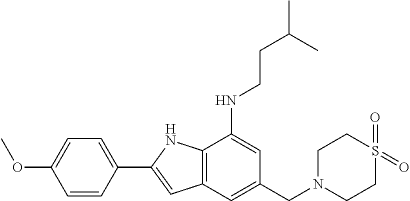 [SMILES: COc1ccc(-c2cc3cc(CN4CCS(=O)(=O)CC4)cc(NCCC(C)C)c3[nH]2)cc1] | 4-((7-(isopentylamino)-2-(4-methoxyphenyl)-1H- indol-5-yl)methyl)thiomorpholine 1,1-dioxide |
| 16 | 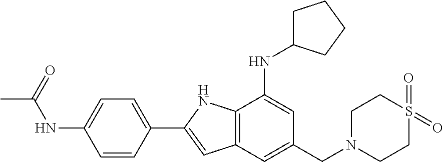 [SMILES: CC(=O)Nc1ccc(-c2cc3cc(CN4CCS(=O)(=O)CC4)cc(NC4CCCC4)c3[nH]2)cc1] | N-(4-(7-(cyclopentylamino)-5-((1,1- dioxidothiomorpholino)methyl)-1H-indole-2- yl)phenyl)acetamide |
| 17 | 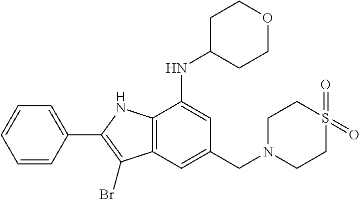 [SMILES: O=S1(=O)CCN(Cc2cc(NC3CCOCC3)c3[nH]c(-c4ccccc4)c(Br)c3c2)CC1] | 4-((3-bromo-2-phenyl-7-((tetrahydro-2H-pyran-4- yl)amino)-1H-indole-5-yl)methyl)thiomorpholine 1,1-dioxide |
| 18 | 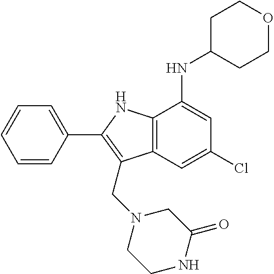 [SMILES: O=C1CN(Cc2c(-c3ccccc3)[nH]c3c(NC4CCOCC4)cc(Cl)cc23)CCN1] | 4-((5-chloro-2-phenyl-7-((tetrahydro-2H-pyran-4- yl)amino)-1H-indol-3-yl)methyl)piperazine-2-one |
| 19 | 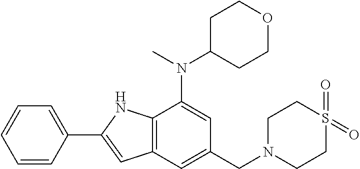 [SMILES: CN(c1cc(CN2CCS(=O)(=O)CC2)cc2cc(-c3ccccc3)[nH]c12)C1CCOCC1] | 4-((7-(methyl(tetrahydro-2H-pyran-4-yl)amino)-2- phenyl-1H-indol-5-yl)methyl)thiomorpholine 1,1- dioxide |
| 20 | 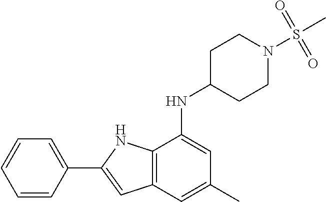 [SMILES: Cc1cc(NC2CCN(S(C)(=O)=O)CC2)c2[nH]c(-c3ccccc3)cc2c1] | 5-methyl-N-(1-(methylsulfonyl)piperidin-4-yl)-2- phenyl-1H-indole-7-amine |
| 21 | 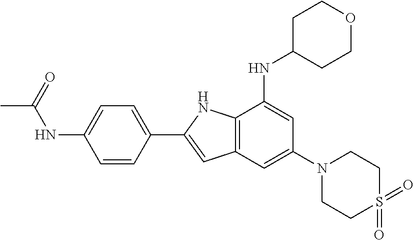 [SMILES: CC(=O)Nc1ccc(-c2cc3cc(N4CCS(=O)(=O)CC4)cc(NC4CCOCC4)c3[nH]2)cc1] | N-(4-(5-(1,1-dioxidothiomorpholino)-7- ((tetrahydro-2H-pyran-4-yl)amino)-1H-indol-2- yl)phenyl)acetamide |
| 22 | 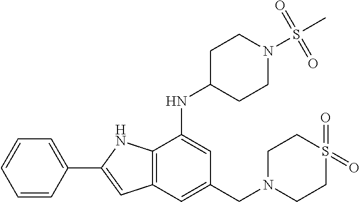 [SMILES: CS(=O)(=O)N1CCC(Nc2cc(CN3CCS(=O)(=O)CC3)cc3cc(-c4ccccc4)[nH]c23)CC1] | 4-((7-((1-(methylsulfonyl)piperidin-4-yl)amino)-2- phenyl-1H-indole-5-yl)methyl)thiomorpholine 1,1- dioxide |
| 23 | 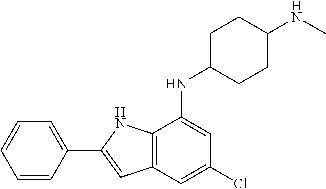 [SMILES: CNC1CCC(Nc2cc(Cl)cc3cc(-c4ccccc4)[nH]c23)CC1] | N1-(5-chloro-2-phenyl-1H-indol-7-yl)-N4- methylcyclohexane-1,4-diamine |
| 24 | 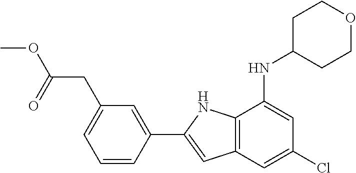 [SMILES: COC(=O)Cc1cccc(-c2cc3cc(Cl)cc(NC4CCOCC4)c3[nH]2)c1] | methyl 2-(3-(5-chloro-7-((tetrahydro-2H-pyran-4- yl)amino)-1H-indol-2-yl)phenyl)acetate |
| 25 | 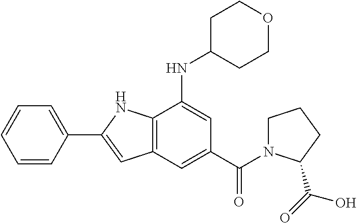 [SMILES: O=C(O)[C@H]1CCCN1C(=O)c1cc(NC2CCOCC2)c2[nH]c(-c3ccccc3)cc2c1] | (2-phenyl-7-((tetrahydro-2H-pyran-4-yl)amino)- 1H-indol-5-carbonyl)-D-proline |
| 26 | 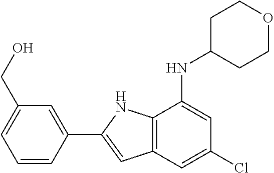 [SMILES: OCc1cccc(-c2cc3cc(Cl)cc(NC4CCOCC4)c3[nH]2)c1] | (3-(5-chloro-7-((tetrahydro-2H-pyran-4-yl)amino)- 1H-indol-2-yl)phenyl)methanol |
| 27 | 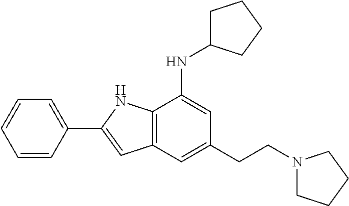 [SMILES: c1ccc(-c2cc3cc(CCN4CCCC4)cc(NC4CCCC4)c3[nH]2)cc1] | N-cyclopentyl-2-phenyl-5-(2-(pyrrolidin-1- yl)ethyl)-1H-indole-7-amine |
| 28 | 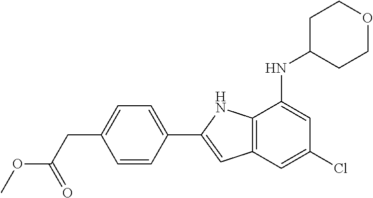 [SMILES: COC(=O)Cc1ccc(-c2cc3cc(Cl)cc(NC4CCOCC4)c3[nH]2)cc1] | methyl 2-(4-(5-chloro-7-((tetrahydro-2H-pyran-4- yl)amino)-1H-indol-2-yl)phenyl)acetate |
| 29 | 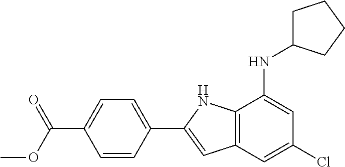 [SMILES: COC(=O)c1ccc(-c2cc3cc(Cl)cc(NC4CCCC4)c3[nH]2)cc1] | methyl 4-(5-chloro-7-(cyclopentylamino)-1H- indol-2-yl)benzoate |
| 30 | 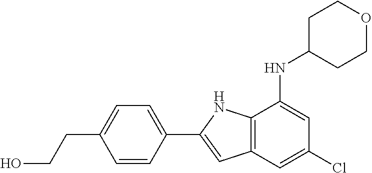 [SMILES: OCCc1ccc(-c2cc3cc(Cl)cc(NC4CCOCC4)c3[nH]2)cc1] | 2-(4-(5-chloro-7-((tetrahydro-2H-pyran-4- yl)amino)-1H-indol-2-yl)phenyl)ethan-1-ol |
| 31 | 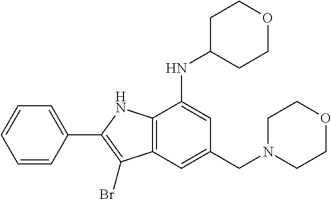 [SMILES: Brc1c(-c2ccccc2)[nH]c2c(NC3CCOCC3)cc(CN3CCOCC3)cc12] | 3-bromo-5-(morpholinomethyl)-2-phenyl-N- (tetrahydro-2H-pyran-4-yl)-1H-indole-7-amine |
| 32 | 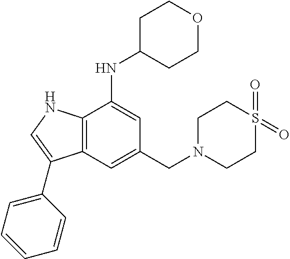 [SMILES: O=S1(=O)CCN(Cc2cc(NC3CCOCC3)c3[nH]cc(-c4ccccc4)c3c2)CC1] | 4-((3-phenyl-7-((tetrahydro-2H-pyran-4- yl)amino)-1H-indol-5-yl)methyl)thiomorpholine 1,1-dioxide |
| 33 | 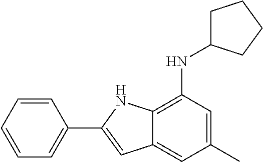 [SMILES: Cc1cc(NC2CCCC2)c2[nH]c(-c3ccccc3)cc2c1] | N-cyclopentyl-5-methyl-2-phenyl-1H-indole-7- amine |

In preferable exemplary embodiments of the present invention, the compound of Chemical Formula 1 may be a compound of the following Chemical Formula 2.

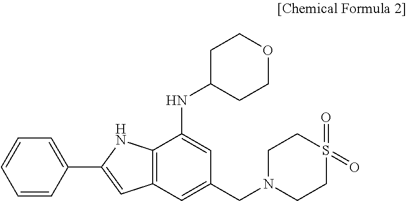 [SMILES: O=S1(=O)CCN(Cc2cc(NC3CCOCC3)c3[nH]c(-c4ccccc4)cc3c2)CC1]

A compound of Chemical Formula 1, an isomer thereof, a solvate thereof, a hydrate thereof, or a pharmaceutically acceptable salt thereof according to the present invention can effectively inhibit ferroptosis among types of cell death.

The mechanism of ferroptosis may be divided into 1) generation of reactive oxygen species, especially lipid reactive oxygen species, 2) reduction of the antioxidant enzyme, glutathione peroxidase 4 (GPX4), 3) accumulation of lipid peroxides, and 4) increase of iron. To confirm the ferroptosis inhibition efficacy of the compound of Chemical Formula 1 in the present invention, ferroptosis was induced in various cells (cardiac cells, adrenal medulla cells, renal fibroblasts, retinal epithelial cells, lung epithelial cells, renal proximal tubule epithelial cells, hippocampal neurons, and fibroblasts), and the effect of protecting against cell death caused by the induced ferroptosis, the effect of inhibiting accumulation of cytosolic lipid reactive oxygen species and mitochondrial reactive oxygen species, the effect of inhibiting accumulation of cytosolic and mitochondrial iron ions, and the effect of inhibiting morphological changes in mitochondria were experimentally confirmed. In particular, the present invention can provide a compound exhibiting a better effect than ferrostatin-1, which is a currently well-known ferroptosis inhibitor, and a better effect, in terms of the effect of inhibiting accumulation of cytosolic and mitochondrial iron ions, than deferoxamine (DFO), which is a reagent chelating iron ions.

Ferroptosis, which is a type of programmed cell death, is clearly distinguished from apoptosis, necroptosis, and pyroptosis.

In apoptosis, cell death occurs through chromatin condensation, DNA fragmentation, plasma membrane blebbing, and increased mitochondrial membrane permeability. In necroptosis, a decrease in cell membrane homeostasis through plasma membrane rupture and expansion of organelles occurs, but no nuclear fragmentation occurs. In pyroptosis, pores are formed in the cell membrane by gasdermin D in a caspase-1-dependent manner, and a pathologically excessive inflow of ions leads to cell lysis, resulting in cell death.

However, ferroptosis is iron-dependent cell death characterized in that the size of mitochondria shrinks, cristae structures in the inner membrane disappear, and the outer mitochondrial membrane ruptures.

Since the mechanism of action of each type of cell death is different, the substances that induce cell death or their inhibitors may also be clearly different depending on the type of cell death. It was confirmed that the compound of Chemical Formula 1 has an inhibitory effect against ferroptosis, which is a type of cell death distinguished from other types of cell death.

Substances used to induce ferroptosis include type 1 and type 2 inducers. Type 1 ferroptosis inducers include erastin, sorafenib, and sulfasalazine, which induce ferroptosis by inhibiting system Xc to prevent cystine inflow, thereby lowering GSH synthesis and GPX4 activity. Type 2 ferroptosis inducers act by directly inhibiting GPX4; RSL3, one of the type 2 ferroptosis inducers, inhibits the enzymatic activity of GPX4 through an irreversible covalent bond with selenocysteine of GPX4. Type 1 and type 2 ferroptosis inducers have a common feature in that they result in inhibiting GPX4 activity, but their mechanisms of action are different. In the examples described below, the present inventors confirmed that the compound of Chemical Formula 1 can inhibit ferroptosis induced by both type 1 and/or type 2 inducers, and therefore, it is thought that the compound of Chemical Formula 1 according to the present invention inhibits ferroptosis without being limited by the method of inducing ferroptosis.

It has been reported that the occurrence of ferroptosis is related to the development mechanism of various cancers and diseases such as Parkinson's disease, sepsis, and liver diseases. Accordingly, the present invention can prevent or treat diseases related to ferroptosis by inhibiting ferroptosis.

The present invention provides a pharmaceutical composition for preventing or treating ferroptosis-related diseases, including a compound of Chemical Formula 1, an isomer thereof, a solvate thereof, a hydrate thereof, or a pharmaceutically acceptable salt thereof; and a pharmaceutically acceptable carrier.

The ferroptosis-related disease may be one or more selected from the group consisting of:
acute or chronic liver diseases, including hepatitis, liver fibrosis, or cirrhosis;
neurodegenerative diseases including dementia including Alzheimer's disease and vascular dementia, Parkinson's disease, epilepsy, dementia with Lewy bodies, or Huntington's disease;
ischemic diseases including ischemic heart disease, reperfusion injury, ischemic stroke, or ischemic injury;
pancreatitis, bacterial or viral sepsis, diabetes or diabetic complications, diabetic vascular diseases;
necrotizing proctitis, cystic fibrosis, rheumatoid arthritis, degenerative arthritis, nephrotic syndrome, bacterial infections, viral infections including SARS-COV, multiple sclerosis, leukemia, lymphoma, neonatal respiratory distress syndrome, asphyxia, tuberculosis, endometriosis, angiasthenia, frostbite, post-steroid injection complications, vibrio vulnificus sepsis, tenderness, hemoglobinuria, burns, hyperthermia, celiac disease, compartment syndrome, spinal cord injury, glomerulonephritis, renal failure, metabolic genetic diseases, mycoplasma infection, anthrax, Fabry-Anderson disease, congenital mitochondrial diseases, phenylketonuria, placental infarction, syphilis, and aseptic necrosis;
necrosis associated with exposure to drugs including antibiotics, anticancer agents, doxorubicin, puromycin, bleomycin, non-steroidal anti-inflammatory drugs (NSAIDs) or cyclosporine, chemical toxins including carbon tetrachloride, cyanide, methanol or ethylene glycol, poisonous gases, pesticides, heavy metals including lead, mercury, or cadmium or administration or self-administration thereof; and
damage caused by radiation or ultraviolet exposure and cell necrosis associated therewith.

In addition, it is anticipated that the compound of Chemical Formula 1 will exhibit preventive, therapeutic, and ameliorating effects on acute or chronic kidney diseases, traumatic brain injury, necrotizing colitis, viral infections including SARS-COV, skin diseases including psoriasis and allergic dermatitis, organ preservation or organ transplantation (see Korean Registered Patents 10-1098583 and 10-1941004) or the like.

In addition, the compound of Chemical Formula 1 may inhibit cell death through ferroptosis due to the accumulation of lipid peroxides caused by the Fenton reaction and GPX4 path disruption due to RSL3, erastin or glutamate, which are ferroptosis-inducing substances.

Therefore, a pharmaceutical composition comprising the compound of Chemical Formula 1 may exhibit preventive, therapeutic, and ameliorating effects on ferroptosis diseases. Related diseases comprise:
acute respiratory distress syndrome or acute lung diseases, pneumonia, tuberculosis, asthma, chronic obstructive pulmonary disease (COPD), chronic inflammatory pulmonary diseases including idiopathic pulmonary fibrosis (IPF) and cystic fibrosis (refer to Mitochondrial dysfunction in fibrotic diseases. Cell Death Discov. 2020 Sep. 5; 6:80; Mitochondrial dysfunction in lung aging and diseases. Eur Respir Rev. 2020 Oct. 15; 29(157): 200165; Korean Registered Patent 10-1636563);
demyelinating diseases including demyelination and amyotrophic lateral sclerosis (ALS), hypertension including pulmonary hypertension, stroke, prion disease, epilepsy, ataxia, migraines, memory and cognitive decline, seizures, tremors, or psychiatric disorders including depression (refer to Neuronal and glial calcium signaling in Alzheimer's disease. Cell Calcium. October-November 2003; 34(4-5): 385-97; Mitochondrial disorders: challenges in diagnosis & treatment. Indian J Med Res. 2015 January; 141(1): 13-26.);
spinocerebellar degeneration including Friedreich's ataxia (refer to Ferroptosis in Friedreich's Ataxia: A Metal-Induced Neurodegenerative Disease. Biomolecules. 2020 Nov. 13; 10(11):1551.; The molecular and metabolic landscape of iron and ferroptosis in cardiovascular disease. Nat Rev Cardiol. 2023 January; 20(1):7-23);
dyslipidemia including insulin resistance and hyperlipidemia, atherosclerosis, inflammatory bowel diseases (IBDs) including Crohn's disease and ulcerative colitis;
various cancers and metastasis of cancer (refer to Reticulum stress and oxidative stress in cell fate decision and human disease. Antioxid Redox Signal. 2014 Jul. 20; 21(3):396-413.);
diseases related to visual impairment, including macular degeneration, retinitis pigmentosa, cataracts, and glaucoma; anemia, cholestasis, hypoparathyroidism, pancytopenia, pancreatic disorders, lactic acidosis, lacticemia, hearing loss, short stature, ileus, and cardiac conduction defects including arrhythmia, cardiomyopathy, myocardial infarction, ischemia-reperfusion heart damage, heart failure, endometriosis, infertility, subfertility, early menopause (refer to Mitochondrial diseases: the contribution of organelle stress responses to pathology. Nat Rev Mol Cell Biol. 2018 February; 19(2): 77-92. Seminars in medicine of the Beth Israel Hospital, Boston; Mitochondrial DNA and disease. N Engl J Med. 1995 Sep. 7; 333(10):638-44; Mitochondrial injury and dysfunction in hypertension-induced cardiac damage. Eur Heart J. 2014 Dec. 7; 35(46): 3258-3266.);
muscular dystrophy diseases including limb-girdle muscular dystrophy (LGMD), Becker muscular dystrophy (BMD), and Duchenne muscular dystrophy (DMD) (refer to Duchenne muscular dystrophy is associated with the inhibition of calcium uniport in mitochondria and an increased sensitivity of the organelles to the calcium-induced permeability transition. Biochim Biophys Acta Mol Basis Dis. 2020 May 1; 1866(5): 165674.);
aging and age-related diseases (refer to Interrelation between ROS and Ca²⁺ in aging and age-related diseases. Redox Biology. 2020; 6:101678.);
mucositis including oral mucositis and gastrointestinal mucositis (refer to Emerging role of mitochondrial DAMPs, aberrant mitochondrial dynamics and anomalous mitophagy in gut mucosal pathogenesis. Life Sci. 2022 Sep. 15; 305:120753; The Impacts of Iron Overload and Ferroptosis on Intestinal Mucosal Homeostasis and Inflammation, Int J Mol Sci. 2022 Nov. 17; 23(22): 14195.).

In the present invention, specifically, the ferroptosis-related disease may be neurodegenerative diseases, liver diseases, kidney diseases, stroke, myocardial infarction, ocular diseases, lung diseases, or heart diseases.

The neurodegenerative disease may be one or more selected from dementia including Alzheimer's disease, dementia with Lewy bodies, and vascular dementia, Parkinson's disease, epilepsy, Huntington's disease, amyotrophic lateral sclerosis (ALS), Friedreich's ataxia, multiple sclerosis, and Charcot-Marie-Tooth (CMT) disease.

The liver disease may be one or more selected from liver fibrosis, cirrhosis, liver cancer, and inflammatory liver diseases, wherein the inflammatory liver disease may be one or more selected from hepatitis, acute hepatitis, chronic hepatitis, alcoholic hepatitis, non-alcoholic hepatitis, viral hepatitis, toxic liver disease, and liver abscesses, granulomatous hepatitis, autoimmune hepatitis, and lupus hepatitis.

The kidney disease may be one or more selected from diabetic kidney disease, nephritis, hypertensive nephropathy, glomerulonephritis, polycystic kidney, urinary tract obstruction, and renal fibrosis.

The ocular disease may be one or more selected from retinopathy, retinitis pigmentosa, optic neuropathy, cataract, glaucoma, ocular inflammation, blepharitis, uveitis, optic neuritis, macular degeneration, retinal detachment, dry eye, retinal vascular disease, and corneal disorders.

The lung diseases may be one or more selected from acute respiratory distress syndrome or acute lung disease, inflammatory lung disease, chronic obstructive pulmonary disease (COPD), idiopathic pulmonary fibrosis (IPF), cystic pulmonary fibrosis, sinusitis, allergic rhinitis, lower respiratory tract infections, acute and chronic bronchitis, emphysema, pneumonia, tuberculosis, asthma, bronchiectasis, sequelae of pulmonary tuberculosis, acute respiratory distress syndrome, and pulmonary fibrosis. (refer to Mitochondrial dysfunction in fibrotic diseases. Cell Death Discov. 2020 Sep. 5; 6:80; Mitochondrial dysfunction in lung aging and diseases Eur Respir Rev. 2020 Oct. 15; 29(157): 200165; Korea Registered Patent 10-1636563).

In the present specification, “treatment” means stopping or delaying the progression of a disease, or reversing or alleviating symptoms thereof when used on a subject exhibiting symptoms of the disease, and “prevention” means stopping or delaying the onset of symptoms when used on a subject exhibiting no symptoms but is at high risk.

In the present invention, a “pharmaceutical composition” may include a pharmaceutically acceptable carrier as needed along with the compound of the present invention.

The compound of Chemical Formula 1 according to the present invention may be administered in various oral and parenteral dosage forms upon clinical administration, and when formulated, it is prepared using commonly used diluent or excipients such as fillers, extending agents, binders, wetting agents, disintegrants, or surfactants.

Solid preparations for oral administration include tablets, pills, powder, granules, capsules, troches, or the like, and these solid preparations are prepared by mixing the compound of the present invention with at least one excipient, for example, starch, calcium carbonate, sucrose or lactose or gelatin or the like. Additionally, in addition to simple excipients, lubricants such as magnesium stearate or talc are also used. Liquid preparations for oral administration include suspensions, solutions for internal use, emulsions, or syrups. In addition to water and liquid paraffin, which are commonly used simple diluents, various excipients, for example, wetting agents, sweeteners, fragrances, preservatives or the like may be included.

Preparations for parenteral administration include sterilized aqueous solutions, non-aqueous solutions, suspensions, emulsions, freeze-dried preparations, and suppositories. As non-aqueous solvents and suspensions, propylene glycol, polyethylene glycol, vegetable oils such as olive oil, injectable ester such as ethyl oleate or the like may be used. As a base for suppositories, Witepsol, macrogol, Tween 61, cacao butter, laurin oil, glycerol, gelatin, or the like may be used.

Another aspect of the present invention provides a method of inhibiting ferroptosis, comprising administering the compound of Chemical Formula 1, an isomer thereof, a solvate thereof, a hydrate thereof, or a pharmaceutically acceptable salt thereof to a subject in need thereof in a pharmaceutically effective amount.

In the present invention, the compound of Chemical Formula 1 may interfere with or inhibit ferroptosis by acting on ferroptosis-inducing substances such as erastin, glutamate, or RSL3, and may inhibit cell death through this.

Still another aspect of the present invention provides a method of preventing or treating a ferroptosis-related disease, comprising administering the compound of Chemical Formula 1, an isomer thereof, a solvate thereof, a hydrate thereof, or a pharmaceutically acceptable salt thereof to a subject in need thereof in a pharmaceutically effective amount.

In the present invention, regarding the ferroptosis-related disease, the above-described information may be applied mutatis mutandis.

In the present invention, the ferroptosis-related disease may be accompanied by lipid peroxidation.

In the present invention, “lipid peroxidation” refers to oxidative decomposition of fats, oils, waxes, sterols, triglycerides, or the like, and lipid peroxidation is considered as one of the main causes of the development of various degenerative diseases.

Yet another aspect of the present invention may provide a method of inhibiting the accumulation of mitochondrial or cytosolic iron ions, comprising administering the compound of Chemical Formula 1, an isomer thereof, a solvate thereof, a hydrate thereof, or a pharmaceutically acceptable salt thereof to a subject in need thereof in a pharmaceutically effective amount.

Iron plays a critical important role in cell survival as it is a factor involved in oxygen transport, DNA biosynthesis, and ATP synthesis. Iron induces lipid peroxidation of saturated fatty acids in the body and generates oxygen radicals along with ATP synthesis in mitochondria. When excessive oxygen radicals are generated during this process, oxidative stress may increase and cell death may occur. The compound of Chemical Formula 1 can prevent the excessive accumulation of mitochondrial or cytosolic iron.

Yet another aspect of the present invention provides a method of reducing cytosolic or mitochondrial reactive oxygen species (ROS), comprising bringing a compound of Chemical Formula 1, an isomer thereof, a solvate thereof, a hydrate thereof, or a pharmaceutically acceptable salt thereof into contact with cells, or administering it to a subject in need thereof in a pharmaceutically effective amount.

In the present invention, “reactive oxygen species (ROS)” refers to chemically active molecules, such as free radicals, containing oxygen, and examples of reactive oxygen species comprise oxygen ions, peroxides, and lipid reactive oxygen species. Ferroptosis is characterized by the rapid accumulation of reactive oxygen species in an iron-dependent manner, and the compound of Chemical Formula 1 may inhibit ferroptosis by reducing reactive oxygen species. Yet another aspect of the present invention may provide a method of inhibiting the accumulation of lipid peroxides, comprising administering the compound of Chemical Formula 1, an isomer thereof, a solvate thereof, a hydrate thereof, or a pharmaceutically acceptable salt thereof to a subject in need thereof in a pharmaceutically effective amount. Here, lipid peroxidation may be defined as above.

Yet another aspect of the present invention may be a method of reducing oxidative stress in cells, comprising bringing a compound of Chemical Formula 1, an isomer thereof, a solvate thereof, a hydrate thereof, or a pharmaceutically acceptable salt thereof into contact with cells, or administering it to a subject in need thereof in a pharmaceutically effective amount.

Yet another aspect of the present invention may be a method of inhibiting morphological changes in mitochondria, comprising bringing a compound of Chemical Formula 1, an isomer thereof, a solvate thereof, a hydrate thereof, or a pharmaceutically acceptable salt thereof into contact with cells, or administering it to a subject in need thereof in a pharmaceutically effective amount.

In the present invention, “administration” means providing a predetermined compound of the present invention to a subject by any suitable method.

In the present invention, the “subject” in need of the administration may include both mammals and non-mammals. Here, examples of the mammals may include humans, non-human primates such chimpanzees or monkeys, and livestock animals such as cows, horses, and sheep or the like, but are not limited thereto.

In addition, an effective dosage of the compound of Chemical Formula 1 of the present invention for the human body may vary depending on the patient's age, weight, sex, dosage form, health conditions, and disease level, and is generally about 0.001 to 100 mg/kg/day, and preferably 0.01 to 35 mg/kg/day. Based on an adult patient whose weight is 70 kg, the dosage is generally 0.07 to 7000 mg/day, preferably 0.7 to 2500 mg/day, and the compound may be administered once a day or several times a day at regular intervals by dividing the dosage depending on the judgment of the physician or pharmacist.

A pharmaceutical compound containing the compound of Chemical Formula 1, an isomer thereof, a solvate thereof, a hydrate thereof, or a pharmaceutically acceptable salt thereof as an active ingredient may be administered as separate therapeutic or used in combined administration with another therapeutic in use.

In the present invention, the “cell” is not limited as long as it is a cell of the subject defined above, and examples include cardiac cells, adrenal medulla cells, renal fibroblasts, retinal epithelial cells, lung epithelial cells, renal proximal tubule epithelial cells, hippocampal neurons, and fibroblasts.

In the present invention, “contact” means bringing the compound and optionally one or more additional therapeutic agents into close proximity to the cells in need of such modulation. This may be accomplished using conventional techniques of drug delivery to the subject or in the in vitro situation by, e.g., providing the compound and optionally other therapeutic agents to a culture media in which the cells are located.

Yet another aspect of the present invention provides a use of the compound of Chemical Formula 1, an isomer thereof, a solvate thereof, a hydrate thereof, or a pharmaceutically acceptable salt thereof in preparation of a ferroptosis inhibitor.

Yet another aspect of the present invention provides a use of the compound of Chemical Formula 1, an isomer thereof, a solvate thereof, a hydrate thereof, or a pharmaceutically acceptable salt thereof in prevention or treatment of a ferroptosis-related disease.

Regarding the use and prevention or treatment method of the present invention, the above description of the pharmaceutical composition may be applied mutatis mutandis.

The numerical values described above in the present specification should be interpreted as including the equivalent ranges unless otherwise specified.

Hereinafter, the present invention will be described in detail through the following experimental examples. However, the following experimental examples merely illustrate the present invention, and the content of the present invention is not limited by the following experimental examples. In addition, since these experimental examples are only for the purpose of helping the understanding of the present invention, the scope of the present invention is not limited thereby in any way.

### EXAMPLES

### Experimental Example 1: Cell Culture and Preparation of Example Compounds

Table 2 shows the cells used in the following experimental examples and the composition of each cell medium. All cell lines were cultured at 37° C. in a 5% CO₂ incubator. All cell lines were cultured without contamination by microorganisms such as Mycoplasma.

TABLE 2

| Cell line | Cell type | Supplier | Medium composition |
|---|---|---|---|
| H9C2 | Cardiac cells | ATCC | DMEM, 10% FBS, 1% PS |
| NRK-49F | Renal fibroblasts | ATCC | DMEM, 5% FBS, 1% PS |
| MLE-12 | Lung epithelial | ATCC | DMEM: F-12, 2% FBS, 1% PS, 0.1% |
| | cells | | Insulin-Transferrin-Selenium, 10 nM |
| | | | Hydrocortisone, 10 nM β-estradiol, 10 mM |
| | | | HEPES, 2 mM L-GlutaMax |
| ARPE-19 | Retinal epithelial | ATCC | DMEM: F-12, 10% FBS, 1% PS |
| | cells | | |
| PC-12 | Adrenal medulla | ATCC | DMEM, 10% FBS, 1% PS |
| | cells | | |
| HK-2 | Renal proximal | ATCC | DMEM: F-12, 10% FBS, 1% PS |
| | tubule epithelial | | |
| | cells | | |
| NIH/3T3 | Fibroblasts | ATCC | DMEM, 10% FBS, 1% PS |
| HT-22 | Hippocampal | EMD | DMEM, 10% FBS, 1% PS |
| | neurons | Millipore | |

The example compounds used in the following experimental examples are the compounds listed in Table 1 above, and the example compounds may be prepared by the preparation method known in WO 2009-025478.

### Experimental Example 2: Effect of Protecting Cardiac Cells, Adrenal Medulla Cells, Renal Fibroblasts, Retinal Epithelial Cells, Lung Epithelial Cells, Renal Proximal Tubule Epithelial Cells, Hippocampal Neurons, and Fibroblasts Against Cell Death

To confirm the protective effect on cardiac cells, adrenal medulla cells, renal fibroblasts, retinal epithelial cells, lung epithelial cells, renal proximal tubule epithelial cells, hippocampal neurons, and fibroblasts, 4×10³ to 1.2×10⁴ cells of each cell type were dispensed into 96-well plates and cultured for 18 to 24 hours. Each well was treated with the example compound, which was diluted so that the final concentration became 0.001, 0.003, 0.01, 0.03, 0.1, 0.3, 1, 3, and 10 μM, and culture was performed for 15 to 20 minutes. RSL3, a ferroptosis-inducing substance, was treated at 0.3 to 3 μM for each cell type as shown in Table 3, and culture was performed for 24 hours. To confirm the cell protecting effect of the example compounds, a cytotoxicity LDH assay kit [Dojindo; CK12] was used to measure the level of extracellular secretion of lactate dehydrogenase (LDH). Briefly, at the time point when the 24-hour treatment of each cell type with RSL3 ended, 70 μL of each supernatant from cell culture medium was taken and transferred to a new 96-well plate. After adding 70 μL/well (the same amount as the sample) of the assay buffer included in the kit, the resulting mixture was wrapped with foil and allowed to react at room temperature for 15 to 20 minutes. After observing the color change of the sample, 35 μL/well of a stop solution was added, and the absorbance value at 490 nm was measured using an iD3 spectrophotometer to calculate the EC₅₀ value. Table 3 shows the cell death protection effect of the example compounds after treating each cell type with RSL3, a ferroptosis-inducing substance.

Table 3. Effect of protecting cardiac cells, adrenal medulla cells, renal fibroblasts, retinal epithelial cells, lung epithelial cells, renal proximal tubule epithelial cells, hippocampal neurons, and fibroblasts against cell death induced by RSL3.

TABLE 3

| Cell line | H9C2 | NRK-49F | MLE-12 | ARPE-19 | PC-12 | HK-2 | HT-22 | NIH/3T3 |
|---|---|---|---|---|---|---|---|---|
| RSL3 | 0.5 | 3 | 1 | 0.3 | 0.3 | 0.3 | 0.3 | 3 |
| treatment | | | | | | | | |
| concentration | | | | | | | | |
| (μM) | | | | | | | | |
| | EC₅₀ (μM) | | | | | | | |
| Fer-1 | B | B | B | B | B | B | B | B |
| Example 1 | B | A | A | A | B | A | A | B |
| Example 2 | A | A | A | A | A | A | A | A |
| Example 6 | A | A | A | A | B | A | B | B |
| Example 22 | B | B | A | A | B | B | B | B |
| Example 33 | A | A | A | A | B | A | A | A |

A indicates EC₅₀ ≤ 0.03 μM,
B indicates 0.03 μM < EC₅₀ ≤ 0.1 μM

As shown in Table 3, the example compounds exhibited cell death protection effects similar to or better than ferrostatin-1, which is a representative ferroptosis inhibitor.

### Experimental Example 3. Effect of Inhibiting Cytosolic Lipid Reactive Oxygen Species (Lipid-ROS) in Lung Epithelial Cells, Hippocampal Neurons, Fibroblasts, Adrenal Medulla Cells, and Retinal Epithelial Cells

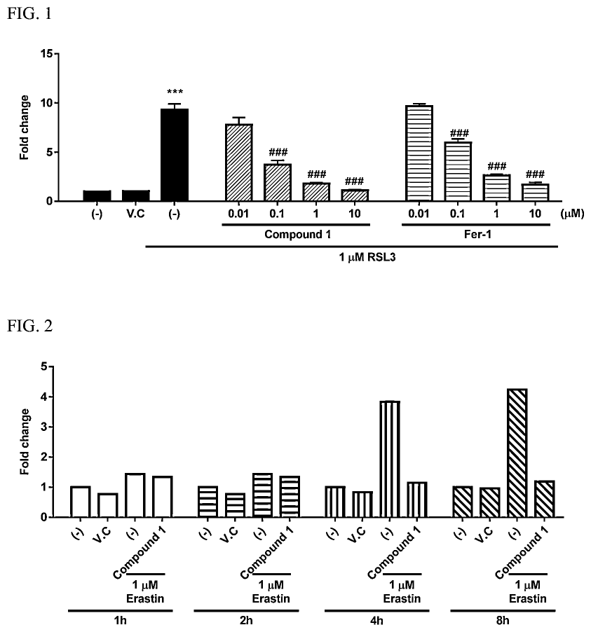

Experimental Example 3-1: To confirm the protective effect against cytosolic lipid reactive oxygen species in lung epithelial cells, 2×10⁵ MLE-12 cells were dispensed into each well of a 12-well plate and cultured for 18 to 24 hours. Each well was treated with Example Compound 1 and ferrostatin-1, which were diluted so that their final concentration became 0.01, 0.1, 1, and 10 μM, and culture was performed for 15 minutes. Afterwards, the cells were treated so that the final concentration of RSL3 became 1 μM, and cultured for 2 hours. To confirm the inhibitory effect against cytosolic lipid reactive oxygen species, BODIPY™ 581/591 C11 (lipid peroxidation sensor) [Invitrogen™; D3861] was used. According to the manufacturer's experimental method, BODIPY™ 581/591 C11 was diluted in the cell culture medium so that the final concentration became 5 μM, and the cells were treated therewith and allowed to react for 30 minutes to perform fluorescent staining. The MLE-12 cells fluorescently stained with BODIPY™ 581/591 C11 were detached from the 12-well plate using 0.25% Trypsin-EDTA and transferred to a microtube, and the fluorescence (excitation/absorption 500 to 650 nm/510 to 665 nm) was measured using BD FACSLyric™ to calculate the rate of change compared to the control group. The results are shown in FIG. 1.

FIG. 1 shows the ratio of cytosolic lipid reactive oxygen species according to each concentration of Example Compound 1 and ferrostatin-1 after treatment with RSL3, a ferroptosis-inducing substance. When the MLE-12 cells were treated with 1 μM RSL3, the cytosolic lipid reactive oxygen species were significantly increased compared to the control group (RSL3 untreated group), and when treated with Example Compound 1 and ferrostatin-1, they were significantly decreased at a concentration of 0.1 μM, and it could be confirmed that the lipid reactive oxygen species decrease as the treatment concentration increases. Therefore, it was confirmed that cytosolic lipid reactive oxygen species increased by RSL3 treatment are effectively inhibited upon treatment with Example Compound 1.

The inhibitory effect against lipid reactive oxygen species was confirmed for HT-22, NIH/3T3, PC-12, and ARPE-19 cells in the same manner as in the above Experimental Example 3-1, except for the different conditions shown in Table 4 below, and the results are shown in FIGS. 2 to 5, respectively.

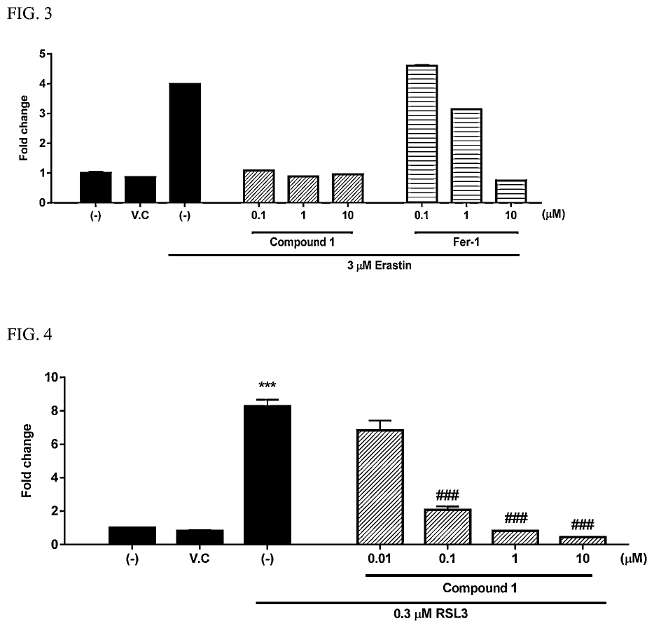

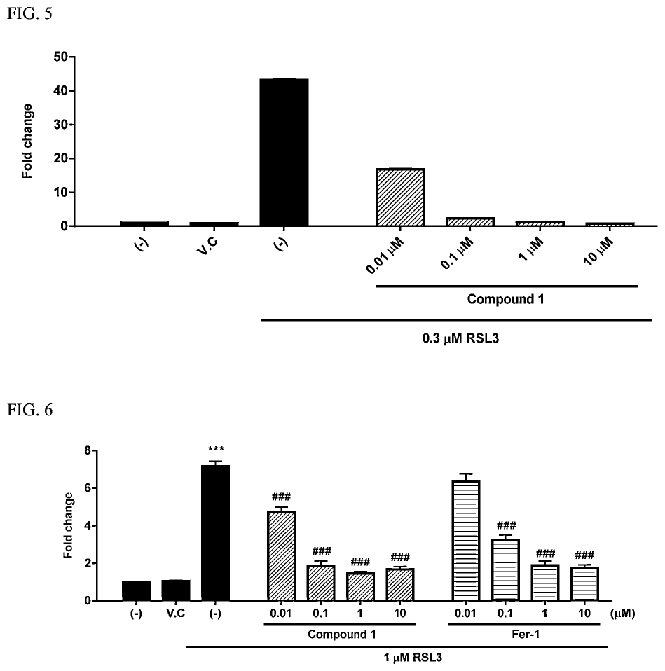

TABLE 4

| | | | | | Example | | | | | |
|---|---|---|---|---|---|---|---|---|---|---|
| | | | | | Compound 1/ | | Ferroptosis | | | |
| | | | | | Ferrostatin-1 | | induction | | | |
| Experi- | Cell line | | | | Treatment | | | Treatment | | FIG. |
| mental | | No. of | No. of | Culture | concentration | Culture | | concentration | Culture | showing |
| Example | Cell | wells | cells | (hr) | (μM) | (min) | Inducer | (μM) | (hr) | results |
| 3-2 | HT-22 | 12 | 1.5 × 10⁵ | 18-24 | 10 | 15 | Erastin | 1 | 1, 2, 4, 8 | FIG. 2 |
| | | | | | — | | | | | |
| 3-3 | NIH/3T3 | 6 | 2 × 10⁵ | 18-24 | 0.1, 1, 10 | 15 | Erastin | 3 | 6 | FIG. 3 |
| | | | | | 0.1, 1, 10 | | | | | |
| 3-4 | PC-12 | 12 | 1.5 × 10⁵ | 18-24 | 0.01, 0.1, | 15 | RSL3 | 0.3 | 1 | FIG. 4 |
| | | | | | 1, 10 | | | | | |
| | | | | | — | | | | | |
| 3-5 | ARPE-19 | 6 | 3 × 10⁵ | 18-24 | 0.01, 0.1, | 20 | RSL3 | 0.3 | 2 | FIG. 5 |
| | | | | | 1, 10 | | | | | |
| | | | | | — | | | | | |

In FIG. 2, it can be confirmed that the cytosolic lipid reactive oxygen species increased by the treatment with erastin (1 μM) in the HT-22 cells were inhibited by treatment with Example Compound 1 at a concentration of 10 μM. In particular, it can be confirmed that the lipid reactive oxygen species were effectively inhibited 4 hours and 8 hours after treatment with Example Compound 1.

FIG. 3 shows that the cytosolic lipid reactive oxygen species increased by the treatment with erastin (3 μM) in the NIH/3T3 cells were decreased by treatment with Example Compound 1 and ferrostatin-1. In particular, when treated with Example Compound 1, the lipid reactive oxygen species were significantly decreased at a concentration of 0.1 μM, confirming that an excellent inhibitory effect was exhibited even at a low concentration. However, since the lipid reactive oxygen species decreased only at a concentration of 10 μM when treated with ferrostatin-1, it was confirmed that Example Compound 1 inhibits lipid reactive oxygen species more effectively than ferrostatin-1.

FIG. 4 shows that the cytosolic lipid reactive oxygen species were increased when PC-12 cells were treated with RSL3 (0.3 μM) and decreased by treatment with Example Compound 1. In particular, a significant decrease was confirmed when the concentration of Example Compound 1 was 0.1 μM, showing that it effectively inhibits lipid reactive oxygen species.

FIG. 5 shows that the cytosolic lipid reactive oxygen species were increased when ARPE-19 cells were treated with RSL3 (0.3 μM) and decreased by treatment with Example Compound 1. In particular, when the concentration of Example Compound 1 was 0.01 μM, it inhibits lipid reactive oxygen species.

### Experimental Example 4: Inhibitory Effect Against Cytosolic Reactive Oxygen Species (Cyto-ROS) in Lung Epithelial Cells

To confirm the protective effect against cytosolic reactive oxygen species in lung epithelial cells, 2×10⁵ MLE-12 cells were dispensed into each well of a 12-well plate and cultured for 18 to 24 hours. Each well was treated with Example Compound 1 and ferrostatin-1, which were diluted so that their final concentration became 0.01, 0.1, 1, and 10 μM, and culture was performed for 15 minutes. Afterwards, the cells were treated so that the final concentration of RSL3 became 1 μM, and cultured for 2 hours. To confirm the inhibitory effect against cytosolic reactive oxygen species, CM-H2DCFDA (general oxidative stress indicator) [Invitrogen™; C6827] was used. According to the manufacturer's experimental method, CM-H2DCFDA was diluted in the cell culture medium so that the final concentration became 5 μM, and the cells were treated therewith and allowed to react for 30 minutes to perform fluorescent staining. The MLE-12 cells fluorescently stained with CM-H2DCFDA were detached from the 12-well plate using 0.25% Trypsin-EDTA and transferred to a microtube, and the fluorescence (excitation/absorption 492 to 495 nm/517 to 527 nm) was measured using BD FACSLyric™ to calculate the rate of change compared to the control group. The results are shown in FIG. 6.

FIG. 6 shows the ratio of cytosolic reactive oxygen species according to each concentration of Example Compound 1 and ferrostatin-1 after treatment with RSL3, a ferroptosis-inducing substance. When the MLE-12 cells were treated with 1 μM RSL3, the cytosolic reactive oxygen species were significantly increased compared to the control group, and a significant decrease was confirmed at a concentration of 0.01 μM upon treatment with Example Compound 1 and at a concentration of 0.1 μM upon treatment with ferrostatin-1. Therefore, it was confirmed that the cytosolic reactive oxygen species increased by RSL3 treatment are more effectively inhibited upon treatment with Example Compound 1 than upon treatment with ferrostatin-1.

### Experimental Example 5: Inhibitory Effect Against Mitochondrial Reactive Oxygen Species (Mito-ROS) in Lung Epithelial Cells

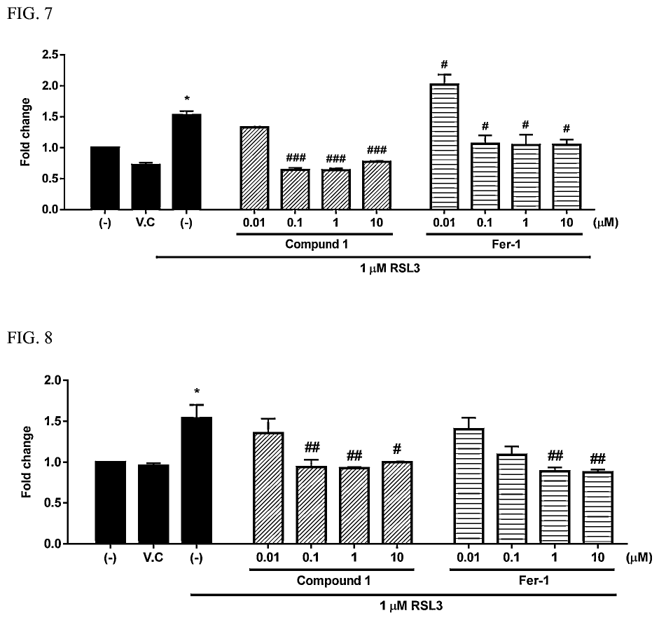

To confirm the protective effect against mitochondrial reactive oxygen species in lung epithelial cells, 2×10⁵ MLE-12 cells were dispensed into each well of a 12-well plate and cultured for 18 to 24 hours. Each well was treated with Example Compound 1 and ferrostatin-1, which were diluted so that their final concentration became 0.01, 0.1, 1, and 10 μM, and culture was performed for 15 minutes. Afterwards, the cells were treated so that the final concentration of RSL3 became 1 μM, and cultured for 2 hours. To confirm the inhibitory effect against mitochondrial reactive oxygen species, MitoSOX™ mitochondrial superoxide indicators (mitochondrial oxidative stress indicator) (MitoSOX) [Invitrogen™; C6827] was used. According to the manufacturer's experimental method, MitoSOX was diluted in the cell culture medium so that the final concentration became 5 μM, and the cells were treated therewith and allowed to react for 30 minutes to perform fluorescent staining. The MLE-12 cells fluorescently stained with MitoSOX were detached from the 12-well plate using 0.25% Trypsin-EDTA and transferred to a microtube, and the fluorescence (excitation/absorption ˜396 nm/610 nm) was measured using BD FACSLyric™ to calculate the rate of change compared to the control group (RSL3 untreated group). The results are shown in FIG. 7.

FIG. 7 shows the ratio of mitochondrial reactive oxygen species according to each concentration of Example Compound 1 and ferrostatin-1 after treatment with RSL3, a ferroptosis-inducing substance. When the MLE-12 cells were treated with 1 μM RSL3, the mitochondrial reactive oxygen species were significantly increased compared to the control group, and when treated with Example Compound 1 and ferrostatin-1, they were significantly decreased at a concentration of 0.1 μM, and it could be confirmed that the mitochondrial reactive oxygen species decrease as the treatment concentration increases. Therefore, it was confirmed that the mitochondrial reactive oxygen species increased by RSL3 treatment are effectively inhibited upon treatment with Example Compound 1.

### Experimental Example 6: Inhibitory Effect Against Cytosolic Iron Ion Accumulation in Lung Epithelial Cells

To confirm the inhibitory effect against cytosolic iron ion accumulation in lung epithelial cells, 2×10⁵ MLE-12 cells were dispensed into each well of a 12-well plate and cultured for 18 to 24 hours. Each well was treated with Example Compound 1 and ferrostatin-1, which were diluted so that their final concentration became 0.01, 0.1, 1, and 10 μM, and culture was performed for 15 minutes. Afterwards, the cells were treated so that the final concentration of RSL3 became 1 μM, and cultured for 8 hours. To confirm the inhibitory effect against cytosolic iron ion accumulation, FerroOrange [DOJINDO; F374] was used. According to the manufacturer's experimental method, FerroOrange was diluted in the cell culture medium so that the final concentration became 1 μM, and the cells were treated therewith and allowed to react for 30 minutes to perform fluorescent staining. The MLE-12 cells fluorescently stained with FerroOrange were detached from the 12-well plate using 0.25% Trypsin-EDTA and transferred to a microtube, and the fluorescence (excitation/absorption 580 nm/543 nm) was measured using BD FACSLyric™ to calculate the rate of change compared to the control group (RSL3 untreated group). The results are shown in FIG. 8.

FIG. 8 shows the ratio of cytosolic iron ion accumulation according to each concentration of Example Compound 1 and ferrostatin-1 after treatment with RSL3, a ferroptosis-inducing substance. When the MLE-12 cells were treated with 1 μM RSL3, cytosolic iron ion accumulation was significantly increased compared to the control group, and a significant decrease was confirmed at a concentration of 0.1 μM upon treatment with Example Compound 1 and at a concentration of 1 μM upon treatment with ferrostatin-1. Therefore, it was confirmed that cytosolic iron ion accumulation increased by RSL3 treatment is more effectively inhibited upon treatment with Example Compound 1 than upon treatment with ferrostatin-1.

### Experimental Example 7: Inhibitory Effect Against Cytosolic Iron Ion Accumulation in Adrenal Medulla Cells and Cardiac Cells Investigated Through Images

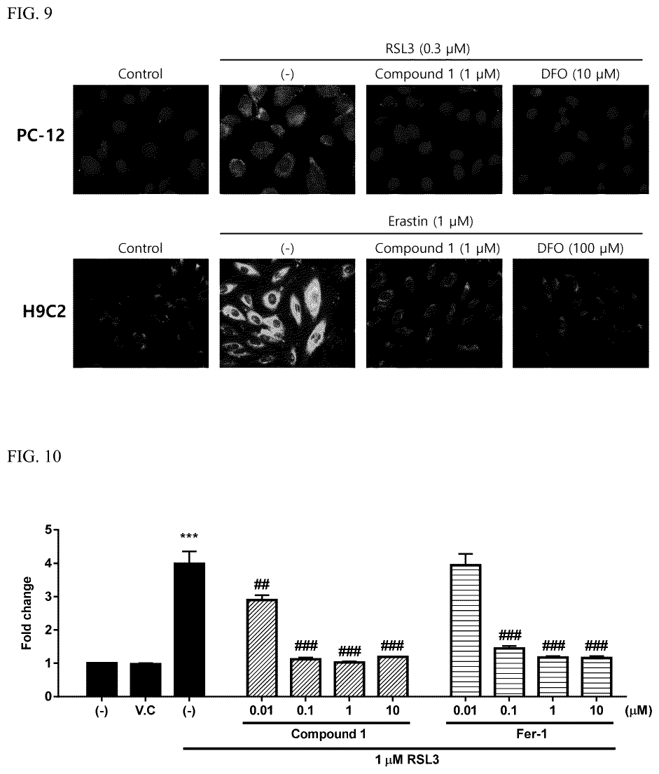

To confirm the inhibitory effect against cytosolic iron ion accumulation in adrenal medulla cells and cardiac cells, 3×10⁵ PC-12 cells and H9C2 cells were each dispensed into a 35 mm confocal dish and cultured for 18 to 36 hours. Each well was treated with the Example Compound 1, which was diluted so that the final concentration became 1 μM, and with DFO, which was diluted so that the final concentration became 10 UM and 100 μM, and culture was performed for 20 to 30 minutes. Afterwards, the PC-12 cells and H9C2 cells were each treated with RSL3 and erastin so that their final concentration became 0.3 UM and 1 μM, respectively, and cultured for 1 hour and 7 hours. To confirm the inhibitory effect against cytosolic iron ion accumulation, FerroOrange [DOJINDO; F374] was used. According to the manufacturer's experimental method, FerroOrange was diluted in the cell culture medium so that the final concentration became 1 μM, and the cells were treated therewith. The PC-12 cells were treated 30 minutes before culture was completed and then fluorescently stained, and the H9C2 cells were treated for 30 minutes after culture was completed and then fluorescently stained. The cells fluorescently stained with FerroOrange were observed using a fluorescence microscope [Carl Zeiss; Axio Observer 7] to investigate the change in fluorescence (excitation/absorption 580 nm/543 nm) compared to the control group (RSL3 and erastin untreated group). The results are shown in FIG. 9.

FIG. 9 shows images of the cytosolic iron ion accumulation according to each concentration of Example Compound 1 and DFO after treatment with RSL3 and erastin, which are ferroptosis-inducing substances. When the PC-12 cells and H9C2 cells were respectively treated with 0.3 μM RSL3 and 1 M erastin, cytosolic iron ion accumulation was significantly increased compared to the control group, and a decrease was confirmed upon treatment with Example Compound 1 (1 μM) and DFO (10 UM or 100 μM). In particular, it can be seen that Example Compound 1 exhibited an effect at a concentration 10 to 100 times lower than that of DFO. Therefore, it was confirmed that the cytosolic iron ion accumulation increased by treatment with RSL3 and erastin is effectively inhibited upon treatment with Example Compound 1.

### Experimental Example 8: Inhibitory Effect Against Mitochondrial Iron Ion Accumulation in Lung Epithelial Cells

To confirm the inhibitory effect against mitochondrial iron ion accumulation in lung epithelial cells, 2×10⁵ MLE-12 cells were dispensed into each well of a 12-well plate and cultured for 18 to 24 hours. Each well was treated with Example Compound 1 and ferrostatin-1, which were diluted so that their final concentration became 0.01, 0.1, 1, and 10 μM, and culture was performed for 15 minutes. Afterwards, the cells were treated so that the final concentration of RSL3 became 1 μM, and cultured for 8 hours. To confirm the inhibitory effect against mitochondrial iron ion accumulation, Mito-FerroGreen [DOJINDO; M489] was used. According to the manufacturer's experimental method, Mito-FerroGreen was diluted in the cell culture medium so that the final concentration became 1 μM, and the cells were treated therewith and allowed to react for 30 minutes to perform fluorescent staining. The MLE-12 cells fluorescently stained with Mito-FerroGreen were detached from the 12-well plate using 0.25% Trypsin-EDTA and transferred to a microtube, and the fluorescence (excitation/absorption 505 nm/535 nm) was measured using BD FACSLyric™ to calculate the rate of change compared to the control group. The results are shown in FIG. 10.

FIG. 10 shows the ratio of mitochondrial iron ion accumulation according to each concentration of Example Compound 1 and ferrostatin-1 after treatment with RSL3, a ferroptosis-inducing substance. When the MLE-12 cells were treated with 1 μM RSL3, mitochondrial iron ion accumulation was significantly increased compared to the control group, and a significant decrease was confirmed at a concentration of 0.01 μM upon treatment with Example Compound 1 and at a concentration of 0.1 μM upon treatment with ferrostatin-1, and it could be confirmed that the mitochondrial iron ion accumulation decreases as the treatment concentration increases. Therefore, it was confirmed that the mitochondrial iron ion accumulation increased by RSL3 treatment is more effectively inhibited upon treatment with Example Compound 1 than upon treatment with ferrostatin-1.

### Experimental Example 9: Inhibitory Effect Against Mitochondrial Iron Ion Accumulation in Lung Epithelial Cells, Adrenal Medulla Cells, and Cardiac Cells Investigated Through Images

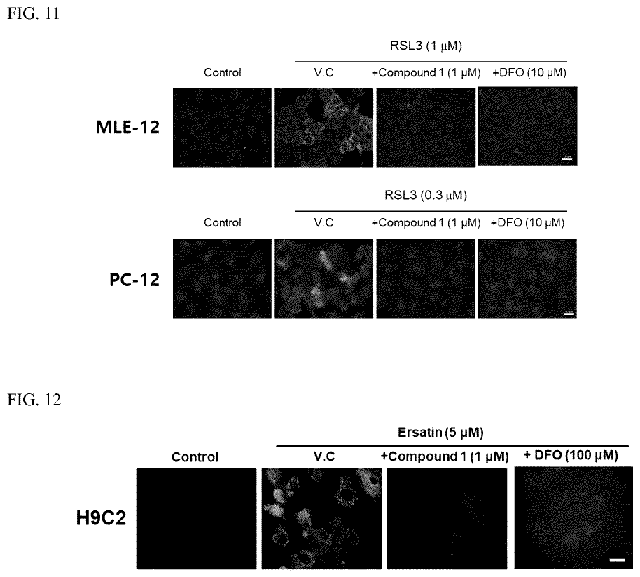

Experimental Example 9-1: To confirm the inhibitory effect against mitochondrial iron ion accumulation in lung epithelial cells and adrenal medulla cells, 3×10⁵ MLE-12 cells and PC-12 cells were each dispensed into a confocal dish and cultured for 18 to 24 hours. Each well was treated with Example Compound 1 and DFO, which were diluted so that their final concentration became 1 μM and 10 μM, respectively, and culture was performed for 20 minutes. Afterwards, the MLE-12 cells were treated with RSL3 so that the final concentration became 1 μM, and cultured for 1 hour and 30 minutes, and the PC-12 cells were treated with RSL3 so that the final concentration became 0.3 μM, and cultured for 30 minutes. To confirm the inhibitory effect against mitochondrial iron ion accumulation, Mito-FerroGreen [DOJINDO; M489] was used. According to the manufacturer's experimental method, Mito-FerroGreen was diluted in the cell culture medium so that the final concentration became 1 μM, and the cells were treated therewith and allowed to react for 30 minutes to perform fluorescent staining. The cells fluorescently stained with Mito-FerroGreen were observed using a fluorescence microscope [Carl Zeiss; Axio Observer 7] to investigate the change in fluorescence (excitation/absorption 450 to 490 nm/500 to 550 nm) compared to the control group (RSL3 untreated group). The results are shown in FIG. 11.

FIG. 11 shows a set of images of an inhibitory effect against mitochondrial iron ion accumulation according to each concentration of Example Compound 1 and DFO after treatment with RSL3, a ferroptosis-inducing substance. When the MLE-12 cells and PC-12 cells were each treated with RSL3, mitochondrial iron ion accumulation was significantly increased compared to the control group, and a decrease was confirmed upon treatment with Example Compound 1 and DFO. In particular, it can be seen that Example Compound 1 exhibited an effect at a concentration 10 times lower than that of DFO. Therefore, it was confirmed that the mitochondrial iron ion accumulation increased by treatment with RSL3 is effectively inhibited upon treatment with Example Compound 1.

The inhibitory effect against mitochondrial iron ion accumulation was confirmed for H9C2 cells in the same manner as in the above Experimental Example 9-1, except for the different conditions shown in Table 5 below, and the results are shown in FIG. 12.

TABLE 5

| | | | | Example | | | | | | |
|---|---|---|---|---|---|---|---|---|---|---|
| | | | | Compound 1/ | | Ferroptosis | | | | |
| | | | | DFO | | induction | | | Mito- | |
| Experi- | Cell line | | | Treatment | | | Treatment | | Ferro | FIG. |
| mental | | No. of | Culture | concentration | Culture | | concentration | Culture | Green | showing |
| Example | Cell | cells | (hr) | (μM) | (min) | Inducer | (μM) | (hr) | (μM) | results |
| 9-2 | H9C2 | 2 × 10⁵ | 18-24 | 1 | 20 | Erastin | 5 | 5 | 1.5 | FIG. 12 |
| | | | | 100 | | | | | | |

FIG. 12 shows a set of images of the mitochondrial iron ion accumulation according to each concentration of Example Compound 1 and DFO after treatment with erastin, a ferroptosis-inducing substance. When the H9C2 cells were treated with erastin, the mitochondrial iron ion accumulation was significantly increased compared to the control group, and the mitochondrial iron ion accumulation was inhibited upon treatment with Example Compound 1 and DFO. In particular, it can be seen that Example Compound 1 exhibited an effect at a concentration 100 times lower than that of DFO. Therefore, it was confirmed that the mitochondrial iron ion accumulation increased by treatment with erastin is effectively inhibited upon treatment with Example Compound 1.

### Experimental Example 10: Cell Death Protection Effect on Cardiac Cells Investigated Through Images

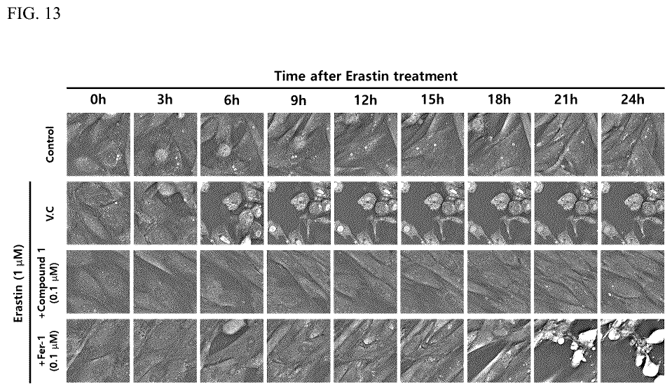

To confirm a cell death protection effect on cardiac cells through images, 2.5×10⁵ H9C2 cells were dispensed into each confocal dish and cultured for 2 days. Each dish was treated with Example Compound 1 and ferrostatin-1, which were diluted to a final concentration of 0.1 μM, and culture was performed for 20 minutes. Afterwards, the cells were treated with erastin so that the final concentration became 1 μM, and images were taken using real-time imaging equipment [Nanolive; CX-A] every 3 hours for 24 hours to confirm changes in the H9C2 cells. The results are shown in FIG. 13.

FIG. 13 shows a set of images of the cell death protection effect of Example Compound 1 and ferrostatin-1 after treatment with erastin, a ferroptosis-inducing substance. In the case where the H9C2 cells were treated with only erastin, cell death was observed after 6 hours. Example Compound 1 inhibited cell death for up to 24 hours, while ferrostatin-1 inhibited cell death for up to 18 hours and cell death was observed from 21 hours. Therefore, it was confirmed that cell death induced by erastin treatment was effectively inhibited by treatment with Example Compound 1 for up to 24 hours and that Example Compound 1 inhibited cell death more effectively than ferrostatin-1.

### Experimental Example 11: Inhibitory Effect Against Mitochondrial Morphological Changes in Cardiac Cells Investigated Through Images

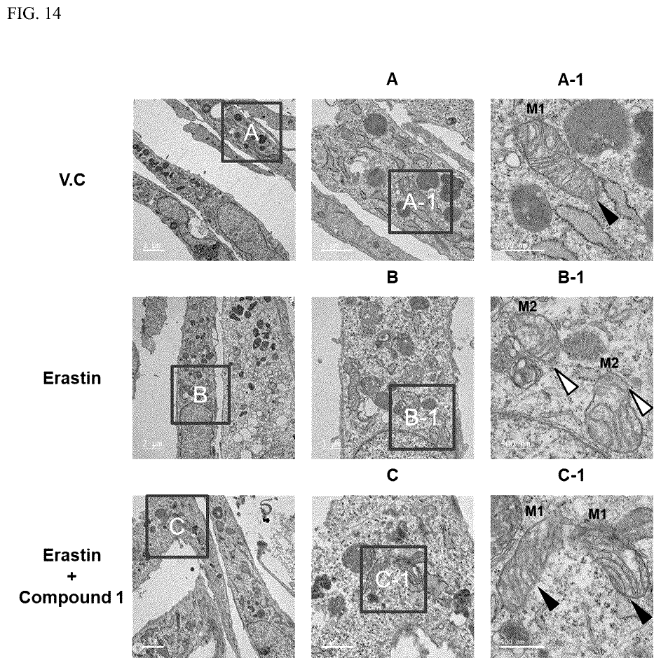

To confirm whether Example Compound 1 may inhibit mitochondrial morphological changes due to ferroptosis, an experiment was performed using a transmission electron microscope (TEM). To collect cardiac cell samples, 1.3×10⁶ H9C2 cells were cultured in a 150 mm cell culture dish for 4 days, and the culture medium was replaced with fresh culture medium on the second day. After treating the cells with Example Compound 1 at a final concentration of 10 UM for 30 minutes, an erastin-only treatment group was treated with a culture medium including 5 μM erastin; an erastin-Example Compound 1 treatment group was treated with a culture medium including 5 μM erastin and 10 μM Example Compound 1; and the V.C group was treated with a culture medium including the same amount of DMSO, and each group was treated for 5 hours. Afterwards, the supernatant was discarded, and the cells were washed with a 37° C. PBS solution three times. 10 mL of a 2.5% glutaraldehyde solution diluted in PBS was added and treated at room temperature for 1 hour. After washing with PBS solution three times, the remaining PBS solution was completely removed using an aspirator. 1 mL of PBS solution was added to recover the cells with a plastic scraper, and the cells were transferred to a 1.5 mL tube. Cells were allowed to settle using a centrifuge (3000 rpm) for 5 minutes. After the supernatant was removed with an aspirator, 1 mL of a 2.5% glutaraldehyde solution was added to perform pre-fixation treatment at 4° C. for 24 hours. The cells were washed three times for 15 minutes each using a 0.1 M cacodylate buffer. A 1% OsO₄ solution diluted in a 0.1 M cacodylate buffer was added to perform post-fixation treatment for 1 hour. The cells were washed twice for 15 minutes each with a 0.1 M cacodylate buffer, and then washed with distilled water for 15 minutes. The distilled water was removed, and the block was stained with 2% uranyl acetate diluted in distilled water for 1 hour. After washing with distilled water three times for 15 minutes each, a dehydration process was performed using ethanol and acetone. The spurr resin was replaced using acetone, and an embedding process was performed at 60° C. for 24 hours. A diamond knife was mounted on an ultra-thin cutter (MTXL, RMC), and ultra-thin sections prepared to have a thickness of 50 to 70 nm were placed on a Cu mesh grid. A post-staining process was performed using 2% uranyl acetate diluted in distilled water for 20 minutes and using citrate for 10 minutes. Mitochondria in the H9C2 cells were observed using a transmission electron microscope (FEI, Tecnai F20 G2). The results are shown in FIG. 14.

FIG. 14 shows the mitochondrial shape of the cardiac cells, H9C2 cells, after treatment with erastin, a ferroptosis-inducing substance, and Example Compound 1. A-1, B-1, and C-1 are enlarged images of A, B and C, and M1 represents mitochondria with a normal structure and M2 represents mitochondria with an abnormal structure. When the H9C2 cells were treated with 5 UM erastin, compared to the control group, changes such as a decrease in size of the mitochondria, disappearance of the cristae structures present in the inner membrane, and collapse of the outer membrane (M2) were observed. However, upon treatment with Example Compound 1 and erastin together, such a phenomenon was alleviated (M1). Therefore, an inhibitory effect of Example Compound 1 on mitochondrial morphological changes under ferroptosis conditions was confirmed.

A compound of Chemical Formula 1, an isomer thereof, a solvate thereof, a hydrate thereof, or a pharmaceutically acceptable salt thereof of the present invention can exhibit an excellent effect of inhibiting or suppressing ferroptosis. In addition, since it exhibits a better effect compared to conventionally known ferroptosis inhibitors, it can be effectively used in treating or preventing diseases caused by ferroptosis.

What is claimed is:

1. A method of inhibiting ferroptosis, comprising administering the compound of Chemical Formula 1, an isomer thereof, a solvate thereof, a hydrate thereof, or a pharmaceutically acceptable salt thereof to a subject in need thereof in a pharmaceutically effective amount: 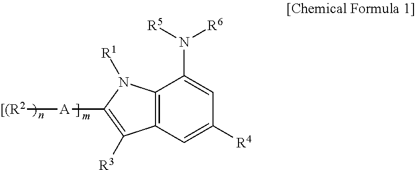
wherein,
n is an integer from 1 to 3,
m is 0 or 1,
A represents phenyl,
R¹ is hydrogen, or a C₁-C₆ alkyl,
R² represents hydrogen, a halogen or a C₁-C₆ alkoxy, or represents hydroxy-C₁-C₆ alkyl, —(CH₂)ₚCO₂R⁷, —NHR⁸, —N(H)S(O)₂R⁷ or —NHC(O)R⁷, wherein p is an integer from 0 to 3, R⁷ represents hydrogen or a C₁-C₃ alkyl, and R⁸ represents (C₁-C₃ alkyl)piperidinyl, or (C₁-C₃ alkyl)sulfonyl,
R³ represents hydrogen, a halogen, a C₁-C₆ alkyl or phenyl, or —(CH₂)p-heterocycle in which the heterocycle is a 5- to 6-membered ring containing one or two heteroatom(s) selected among S, N and O atoms, wherein p is an integer from 0 to 3, provided that R³ is phenyl when m is 0,
R⁴ represents a halogen, a C₁-C₆ alkyl, hydroxy-C₁-C₆ alkyl, —O-phenyl, —(CH₂)ₚCO₂R⁷, —(CH₂)p-heterocycle in which the heterocycle is a 5- to 6-membered ring containing one or two heteroatom(s) selected among S, N and O atoms, or proline-N-carbonyl, wherein p is an integer from 0 to 3, and R⁷ is as described above,
R⁵ is hydrogen, or a C₁-C₆ alkyl, and
R⁶ represents a C₁-C₆ alkyl, a C₃-C₆ cycloalkyl, a heterocycle or heterocyclyl-C₁-C₆ alkyl, wherein the heterocycle is a 3- to 8-membered ring including 1 to 3 heteroatom(s) selected among S, N and O atoms, and R⁶ is optionally substituted with (C₁-C₆ alkyl)amine, hydroxy-C₁-C₆ alkyl, or (C₁-C₆ alkyl)sulfonyl.

2. The method of claim 1, wherein R³ represents hydrogen, a halogen, or phenyl, or represents —(CH₂)p-heterocycle in which the heterocycle is morpholino or piperazinonyl, wherein p is an integer from 0 to 1, provided that R³ is phenyl, when m is 0,
R⁴ is a halogen, a C₁-C₃ alkyl, hydroxy-C₁-C₃ alkyl, —O-phenyl, —(CH₂)ₚCO₂-ethyl, —(CH₂)p-heterocycle in which the heterocycle is thiomorpholino, morpholino, piperazinonyl, or pyrrolidinyl, or proline-N-carbonyl, wherein p is an integer from 0 to 1,
R⁵ is hydrogen, or a C₁-C₃ alkyl, and
R⁶ represents a C₁-C₃ alkyl, a C₃-C₆ cycloalkyl, a heterocycle or heterocyclyl-C₁-C₃ alkyl, wherein the heterocycle is tetrahydro-2H-pyran, or piperidinyl, and when R⁶ is a heterocycle or heterocyclyl-C₁-C₃ alkyl, R⁶ is optionally substituted with (C₁-C₆ alkyl)amine, hydroxy-C₁-C₆ alkyl, or (C₁-C₆ alkyl)sulfonyl.

3. The method of claim 1, wherein the compound of Chemical Formula 1 is any one selected from the following compound group.
<1> 5-[(1,1-dioxido-4-thiomorpholinyl)methyl]-2-phenyl-N-(tetrahydro-2H-pyran-4-yl)-1H-indole-7-amine; <2> ethyl 7-(cyclopentylamino)-2-phenyl-1H-indole-5-carboxylate; <3> (7-(cyclopentylamino)-2-phenyl-1H-indol-5-yl)methanol; <4> 5-chloro-N,1-dimethyl-2-phenyl-N-(tetrahydro-2H-pyran-4-yl)-1H-indole-7-amine; <5> 4-((7-(cyclopentylamino)-2-(3-fluorophenyl)-1H-indol-5-yl)methyl)piperazine-2-one; <6> 4-((2-phenyl-7-(((tetrahydro-2H-pyran-4-yl)methyl)amino)-1H-indol-5-yl)methyl)thiomorpholine 1,1-dioxide; <7> 5-chloro-N-(1-methylpiperidin-4-yl)-2-phenyl-1H-indole-7-amine; <8> 5-phenoxy-2-phenyl-N-(tetrahydro-2H-pyran-4-yl)-1H-indole-7-amine; <9> 5-chloro-3-(morpholinomethyl)-2-phenyl-N-(tetrahydro-2H-pyran-4-yl)-1H-indole-7-amine; <10> 2-(4-((5-fluoro-2-phenyl-1H-indol-7-yl)amino)piperidin-1-yl)ethan-1-ol; <11> N-(4-(5-chloro-7-(cyclopentylamino)-1H-indol-2-yl)phenyl)methanesulfonamide; <12> 5-chloro-3-phenyl-N-(tetrahydro-2H-pyran-4-yl)-1H-indole-7-amine; <13> 4-((2-(3-fluorophenyl)-7-((tetrahydro-2H-pyran-4-yl)amino)-1H-indol-5-yl)methyl)thiomorphline 1,1-dioxide; <14> 5-chloro-N-cyclopentyl-2-(4-((1-methylpiperidin-4-yl)amino)phenyl)-1H-indole-7-amine; <15> 4-((7-(isopentylamino)-2-(4-methoxyphenyl)-1H-indol-5-yl)methyl)thiomorpholine 1,1-dioxide; <16> N-(4-(7-(cyclopentylamino)-5-((1,1-dioxidothiomorpholino)methyl)-1H-indol-2-yl)phenyl)acetamide; <17> 4-((3-bromo-2-phenyl-7-((tetrahydro-2H-pyran-4-yl)amino)-1H-indol-5-yl)methyl)thiomorpholine 1,1-dioxide; <18> 4-((5-chloro-2-phenyl-7-((tetrahydro-2H-pyran-4-yl)amino)-1H-indol-3-yl)methyl)piperazine-2-one; <19> 4-((7-(methyl(tetrahydro-2H-pyran-4-yl)amino)-2-phenyl-1H-indol-5-yl)methyl)thiomorpholine 1,1-dioxide; <20> 5-methyl-N-(1-(methylsulfonyl)piperidin-4-yl)-2-phenyl-1H-indole-7-amine; <21> N-(4-(5-(1,1-dioxidothiomorpholino)-7-((tetrahydro-2H-pyran-4-yl)amino)-1H-indol-2-yl)phenyl)acetamide; <22> 4-((7-((1-(methylsulfonyl)piperidin-4-yl)amino)-2-phenyl-1H-indole-5-yl)methyl)thiomorpholine 1,1-dioxide; <23> N1-(5-chloro-2-phenyl-1H-indol-7-yl)-N4-methylcyclohexane-1,4-diamine; <24> methyl 2-(3-(5-chloro-7-((tetrahydro-2H-pyran-4-yl)amino)-1H-indol-2-yl)phenyl)acetate; <25> (2-phenyl-7-((tetrahydro-2H-pyran-4-yl)amino)-1H-indol-5-carbonyl)-D-proline; <26> (3-(5-chloro-7-((tetrahydro-2H-pyran-4-yl)amino)-1H-indol-2-yl)phenyl)methanol; <27> N-cyclopentyl-2-phenyl-5-(2-(pyrrolidin-1-yl)ethyl)-1H-indole-7-amine; <28> methyl 2-(4-(5-chloro-7-((tetrahydro-2H-pyran-4-yl)amino)-1H-indol-2-yl)phenyl)acetate; <29> methyl 4-(5-chloro-7-(cyclopentylamino)-1H-indol-2-yl)benzoate; <30> 2-(4-(5-chloro-7-((tetrahydro-2H-pyran-4-yl)amino)-1H-indol-2-yl)phenyl)ethan-1-ol; <31> 3-bromo-5-(morpholinomethyl)-2-phenyl-N-(tetrahydro-2H-pyran-4-yl)-1H-indole-7-amine; <32> 4-((3-phenyl-7-((tetrahydro-2H-pyran-4-yl)amino)-1H-indol-5-yl)methyl)thiomorpholine 1,1-dioxide; and <33> N-cyclopentyl-5-methyl-2-phenyl-1H-indole-7-amine.

4. The method of claim 1, wherein the compound of Chemical Formula 1 is a compound of the following Chemical Formula 2.

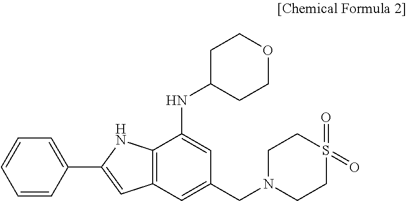 [SMILES: O=S1(=O)CCN(Cc2cc(NC3CCOCC3)c3[nH]c(-c4ccccc4)cc3c2)CC1]

5. The method according to claim 1, wherein ferroptosis inhibition is preventing or treating a ferroptosis-related disease selected from:
acute or chronic liver diseases, including hepatitis, liver fibrosis, or cirrhosis; neurodegenerative diseases including dementia including Alzheimer's disease and vascular dementia, Parkinson's disease, epilepsy, or Huntington's disease; ischemic diseases including ischemic heart disease, reperfusion injury, ischemic stroke, or ischemic injury; pancreatitis, bacterial or viral sepsis, diabetes or diabetic complications, diabetic vascular diseases; necrotizing proctitis, cystic fibrosis, rheumatoid arthritis, degenerative arthritis, nephrotic syndrome, bacterial infections, viral infections including SARS-CoV, multiple sclerosis, leukemia, lymphoma, neonatal respiratory distress syndrome, asphyxia, tuberculosis, endometriosis, angiasthenia, frostbite, post-steroid injection complications, vibrio vulnificus sepsis, tenderness, hemoglobinuria, burns, hyperthermia, celiac disease, compartment syndrome, spinal cord injury, glomerulonephritis, renal failure, metabolic genetic diseases, mycoplasma infection, anthrax, Fabry-Anderson disease, congenital mitochondrial diseases, phenylketonuria, placental infarction, syphilis, and aseptic necrosis; alcoholism and cocaine addiction; necrosis associated with exposure to drugs including antibiotics, anticancer agents, doxorubicin, puromycin, bleomycin, non-steroidal anti-inflammatory drugs (NSAIDs) or cyclosporine, chemical toxins including carbon tetrachloride, cyanide, methanol or ethylene glycol, poisonous gases, pesticides, heavy metals including lead, mercury, or cadmium or administration or self-administration thereof; damage caused by radiation or ultraviolet exposure and cell necrosis associated therewith; acute or chronic kidney diseases, traumatic brain injury, necrotizing colitis, skin diseases including psoriasis and allergic dermatitis; organ preservation or organ transplantation; acute respiratory distress syndrome or acute lung diseases, pneumonia, tuberculosis, asthma, chronic obstructive pulmonary disease (COPD), chronic inflammatory pulmonary diseases including idiopathic pulmonary fibrosis (IPF) and cystic fibrosis; demyelinating diseases including demyelination and amyotrophic lateral sclerosis (ALS); hypertension including pulmonary hypertension; stroke, prion disease, epilepsy, ataxia, migraines, memory and cognitive decline, seizures, tremors, or psychiatric disorders including depression; spinocerebellar degeneration including Friedreich's ataxia; dyslipidemia including insulin resistance and hyperlipidemia, atherosclerosis, inflammatory bowel diseases (IBDs) including Crohn's disease and ulcerative colitis; various cancers and metastasis of cancer; diseases related to visual impairment including macular degeneration, retinitis pigmentosa, cataracts, or glaucoma; anemia, cholestasis, hypoparathyroidism, pancytopenia, pancreatic disorders, lactic acidosis, lacticemia, hearing loss, short stature, ileus, cardiac conduction defects including arrhythmia, cardiomyopathy, myocardial infarction, ischemia-reperfusion heart damage, heart failure, endometriosis, infertility, subfertility, early menopause; muscular dystrophy diseases including limb-girdle muscular dystrophy (LGMD), Becker muscular dystrophy (BMD), and Duchenne muscular dystrophy (DMD); age-related diseases; mucositis including oral mucositis and gastrointestinal mucositis.

6. The method according to claim 1, wherein ferroptosis inhibition is preventing or treating neurodegenerative diseases, liver diseases, kidney diseases, stroke, myocardial infarction, ocular diseases, lung diseases, or heart diseases.

7. The method according to claim 6, wherein the neurodegenerative diseases are one or more selected from dementia including Alzheimer's disease, dementia with Lewy bodies, and vascular dementia, Parkinson's disease, epilepsy, Huntington's disease, amyotrophic lateral sclerosis. (ALS), Friedreich's ataxia, multiple sclerosis, and Charcot-Marie-Tooth (CMT) disease.

8. A method of reducing cytosolic or mitochondrial reactive oxygen species (ROS), comprising bringing a compound of Chemical Formula 1, an isomer thereof, a solvate thereof, a hydrate thereof, or a pharmaceutically acceptable salt thereof into contact with cells: 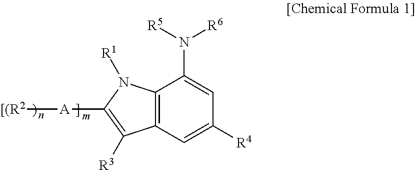
wherein R¹ to R⁶, A, n, and m are each the same as defined in claim 1.

9. A method of inhibiting cytosolic or mitochondrial accumulation of iron ions, comprising administering a compound of Chemical Formula 1, an isomer thereof, a solvate thereof, a hydrate thereof, or a pharmaceutically acceptable salt thereof to a subject in need thereof in a pharmaceutically effective amount: 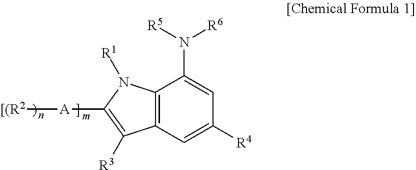
wherein R¹ to R⁶, A, n, and m are each the same as defined in claim 1.
